SEQUENCE LISTING (37 sequences)

SEQ ID NO: 1 - RNA, 22 nt, Homo sapiens
uggaauguaaagaaguauguau

SEQ ID NO: 2 - DNA, 22 bp, Artificial Sequence
  Synthetic
tggaatgtaaagaagtatgtat

SEQ ID NO: 3 - DNA, 22 bp, Artificial Sequence
  Synthetic
  misc_feature (1)..(1): 5′-Amino Modifier C12 modification, e.g., NH2(CH2)12PO4-
atacatacttctttacattcca

SEQ ID NO: 4 - RNA, 22 nt, Homo sapiens
auaagacgaacaaaagguuugu

SEQ ID NO: 5 - DNA, 22 bp, Artificial Sequence
  Synthetic
ataagacgaacaaaaggtttgt

SEQ ID NO: 6 - DNA, 22 bp, Artificial Sequence
  Synthetic
  misc_feature (1)..(1): 5′-Amino Modifier C12 modification, e.g., NH2(CH2)12PO4-
acaaaccttttgttggtcttat

SEQ ID NO: 7 - RNA, 21 nt, Homo sapiens
uuaagacuugcagugauguuu

SEQ ID NO: 8 - DNA, 21 bp, Artificial Sequence
  Synthetic
ttaagacttgcagtgatgttt

SEQ ID NO: 9 - DNA, 21 bp, Artificial Sequence
  Synthetic
  misc_feature (1)..(1): 5′-Amino Modifier C12 modification, e.g., NH2(CH2)12PO4-
aaacatcactgcaagtcttaa

SEQ ID NO: 10 - RNA, 22 nt, Homo sapiens
uaauacugccugguaaugauga

SEQ ID NO: 11 - DNA, 22 bp, Artificial Sequence
  Synthetic
taatactgcctggtaatgatga

SEQ ID NO: 12 - DNA, 22 bp, Artificial Sequence
  Synthetic
  misc_feature (1)..(1): 5′-Amino Modifier C12 modification, e.g., NH2(CH2)12PO4-
tcatcattaccaggcagtatta

SEQ ID NO: 13 - RNA, 22 nt, Homo sapiens
aaacauucgcggugcacuucuu

SEQ ID NO: 14 - DNA, 22 bp, Artificial Sequence
  Synthetic
aaacattcgcggtgcacttctt

SEQ ID NO: 15 - DNA, 22 bp, Artificial Sequence
  Synthetic
  misc_feature (1)..(1): 5′-Amino Modifier C12 modification, e.g., NH2(CH2)12PO4-
aagaagtgcaccgcgaatgttt

SEQ ID NO: 16 - RNA, 21 nt, Homo sapiens
gccccugggccuauccuagaa

SEQ ID NO: 17 - DNA, 21 bp, Artificial Sequence
  Synthetic
gcccctgggcctatcctagaa

SEQ ID NO: 18 - DNA, 21 bp, Artificial Sequence
  Synthetic
  misc_feature (1)..(1): 5′-Amino Modifier C12 modification, e.g., NH2(CH2)12PO4-
ttctaggataggcccaggggc

SEQ ID NO: 19 - RNA, 23 nt, Homo sapiens
ugaggauggauagcaaggaagcc

SEQ ID NO: 20 - DNA, 23 bp, Artificial Sequence
  Synthetic
tgaggatggatagcaaggaagcc

SEQ ID NO: 21 - DNA, 23 bp, Artificial Sequence
  Synthetic
  misc_feature (1)..(1): 5′-Amino Modifier C12 modification, e.g., NH2(CH2)12PO4-
ggcttccttgctatccatcctca

SEQ ID NO: 22 - RNA, 23 nt, Homo sapiens
cagugcaauaguauugucaaagc

SEQ ID NO: 23 - DNA, 23 bp, Artificial Sequence
  Synthetic
cagtgcaatagtattgtcaaagc

SEQ ID NO: 24 - DNA, 23 bp, Artificial Sequence
  Synthetic
  misc_feature (1)..(1): 5′-Amino Modifier C12 modification, e.g., NH2(CH2)12PO4-
gctttgacaatactattgcactg

SEQ ID NO: 25 - RNA, 23 nt, Homo sapiens
uaaggugcaucuagugcagauag

SEQ ID NO: 26 - DNA, 23 bp, Artificial Sequence
  Synthetic
taaggtgcatctagtgcagatag

SEQ ID NO: 27 - DNA, 23 bp, Artificial Sequence
  Synthetic
  misc_feature (1)..(1): 5′-Amino Modifier C12 modification, e.g., NH2(CH2)12PO4-
ctatctgcactagatgcacctta

SEQ ID NO: 28 - RNA, 23 nt, Homo sapiens
ugaggggcagagagcgagacuuu

SEQ ID NO: 29 - DNA, 23 bp, Artificial Sequence
  Synthetic
tgaggggcagagagcgagacttt

SEQ ID NO: 30 - DNA, 23 bp, Artificial Sequence
  Synthetic
  misc_feature (1)..(1): 5′-Amino Modifier C12 modification, e.g., NH2(CH2)12PO4-
aaagtctcgctctctgcccctca

SEQ ID NO: 31 - RNA, 21 nt, Homo sapiens
cauaaaguagaaagcacuacu

SEQ ID NO: 32 - DNA, 21 bp, Artificial Sequence
  Synthetic
cataaagtagaaagcactact

SEQ ID NO: 33 - DNA, 21 bp, Artificial Sequence
  Synthetic
  misc_feature (1)..(1): 5′-Amino Modifier C12 modification, e.g., NH2(CH2)12PO4-
agtagtgctttctactttatg

SEQ ID NO: 34 - RNA, 22 nt, Homo sapiens
uaacagucuacagccauggucg

SEQ ID NO: 35 - DNA, 22 bp, Artificial Sequence
  Synthetic
taacagtctacagccatggtcg

SEQ ID NO: 36 - DNA, 22 bp, Artificial Sequence
  Synthetic
  misc_feature (1)..(1): 5′-Amino Modifier C12 modification, e.g., NH2(CH2)12PO4-
cgaccatggctgtagactgtta

SEQ ID NO: 37 - RNA, 21 nt, Arabidopsis thaliana
uuuggauugaagggagcucua

(19) United States
(12) Patent Application Publication
(10) Pub. No.: US 2024/0191279 A1
(43) Pub. Date: Jun. 13, 2024

(54) METHOD AND DEVICE FOR DETECTION OF MYOCARDIAL INFARCTION AND REPERFUSION INJURY

(71) Applicant: UNIVERSITY OF NOTRE DAME DU LAC, South Bend, IN (US)

(72) Inventors: Hsueh-Chia CHANG, South Bend, IN (US); Pinar ZORLUTUNA, South Bend, IN (US); Satyajyoti SENAPATI, South Bend, IN (US); Xiang REN, South Bend, IN (US)

(21) Appl. No.: 18/556,228

(22) PCT Filed: Apr. 22, 2022
(86) PCT No.: PCT/US2022/025911

§ 371 Date: Oct. 19, 2023

Related U.S. Application Data
(60) Provisional application No. 63/179,313, filed on Apr. 24, 2021.

(51) Int. Cl. C12Q 1/6816

(52) U.S. Cl. CPC C12Q 1/6816; C12Q 2600/112; C12Q 2600/178

(57) ABSTRACT

Described herein are methods and devices for rapid detection of miRNA and protein biomarkers for diagnosing myocardial infarction and reperfusion injury.

### CROSS-REFERENCE TO RELATED APPLICATIONS

This application claims priority to U.S. Provisional Patent Application No. 63/179,313, filed on Apr. 24, 2021, which is incorporated by reference herein in its entirety.

### FEDERALLY SPONSORED RESEARCH

This invention was made with government support under grant number HL141909 awarded by the National Institutes of Health. The government has certain rights in the invention.

### REFERENCE TO SEQUENCE LISTING

This application is filed with a Computer Readable Form of a Sequence Listing in accord with 37 C.F.R. § 1.821(c). The text file submitted by EFS, “092012-9147-WO01_sequence listing_20-APR-2022_ST25.txt,” was created on Apr. 20, 2022, contains 37 sequences, has a file size of 7.86 Kbytes, and is hereby incorporated by reference in its entirety.

### TECHNICAL FIELD

Described herein are methods and devices for rapid detection of miRNA and protein biomarkers for diagnosing myocardial infarction and reperfusion injury.

### BACKGROUND

Acute myocardial infarction (AMI) is the primary cause of death among cardiovascular diseases. The diagnosis of AMI requires accurate analytical results from biomarkers in a timely manner. The current clinical standard of diagnosis combines echocardiogram (ECG) and several circulating protein biomarkers from plasma. Though ECG results can be retrieved within minutes of patient admission, biomarker results typically take hours after blood sampling because of the various pretreatment and reverse-transcription PCR steps. These pretreatment steps also have low yield, thus compromising the sensitivity of the test. In their current state, both are incapable of distinguishing between patients with and without complete coronary occlusion, unless additional invasive testing is implemented, and both have significant false positive rates. These issues prolong diagnosis and treatment, contributing to increased mortality rates.

Thus, there is an unmet clinical need for new approaches for diagnosing AMI and the status of coronary occlusion in a more accurate and rapid manner. In particular, rapid and accurate differentiation between ischemia in the presence or absence of reperfusion injury has important clinical diagnostic and treatment implications.

### SUMMARY

One embodiment described herein is a method for detecting microRNAs (miRNAs) associated with acute myocardial infarction reperfusion injury, or coronary artery disease, the method comprising: obtaining a biological sample from a subject; and performing an assay on the biological sample to detect one or more miRNAs selected from miR-1-1 (SEQ ID NO: 1); miR-208b (SEQ ID NO: 4); miR-499 (SEQ ID NO: 7); miR-200b (SEQ ID NO: 10); miR-543 (SEQ ID NO: 13); miR-331 (SEQ ID NO: 16); miR-3605 (SEQ ID NO: 19); miR-301a (SEQ ID NO: 22); miR-18a (SEQ ID NO: 25); miR-423 (SEQ ID NO: 28); miR-142 (SEQ ID NO: 31); or miR-132 (SEQ ID NO: 34) by hybridization with oligonucleotide probes complementary to the one or more miRNAs. In one aspect, the method can distinguish between acute myocardial infarction, reperfusion injury, coronary artery disease, or normal subjects. In another aspect, the biological sample is selected from one or more of blood, serum, or plasma. In another aspect, the miRNA is free, contained in exosomes, or a combination thereof. In another aspect, the method further comprises determining the concentration of the miRNAs in the biological sample and the miRNA concentrations of a control or normal subject. In another aspect, when the concentration of the miRNA in the biological sample is increased by 10-100-fold as compared to the control or normal subject, the subject is administered a treatment.

Another embodiment described herein is a method for differentiation between acute myocardial infarction reperfusion injury, or coronary artery disease, the method comprising: obtaining a biological sample from a subject; and performing an assay on the biological sample to detect one or more miRNAs selected from miR-1-1 (SEQ ID NO: 1); miR-208b (SEQ ID NO: 4); miR-499 (SEQ ID NO: 7); miR-200b (SEQ ID NO: 10); miR-543 (SEQ ID NO: 13); miR-331 (SEQ ID NO: 16); miR-3605 (SEQ ID NO: 19); miR-301a (SEQ ID NO: 22); miR-18a (SEQ ID NO: 25); miR-423 (SEQ ID NO: 28); miR-142 (SEQ ID NO: 31); or miR-132 (SEQ ID NO: 34) by hybridization with oligonucleotide probes complementary to the one or more miRNAs. In one aspect, the biological sample is selected from one or more of blood, serum, or plasma. In another aspect, the miRNA is free, contained in exosomes, or a combination thereof. In another aspect, the method further comprises determining the concentration of the miRNAs in the biological sample and the miRNA concentrations of a control or normal subject. In another aspect, when the concentration of the miRNA in the biological sample is increased by 10-100-fold as compared to the control or normal subject, the subject is administered a treatment. In another aspect, the treatment comprises administering reperfusion therapy or a therapeutic selected from antiplatelet drugs, anticoagulants, nitrates, beta-blockers, statins, angiotensin-converting enzyme (ACE) inhibitors, angiotensin II receptor blockers (ARBs), calcium channel blockers, therapeutic hypothermia, free radical scavenger drugs, antioxidant drugs or vitamins, anesthesia, bile pigments, hydrogen (H₂), hydrogen sulfide (H₂S), nitrous oxide (NO), carbon monoxide (CO), or a combination thereof.

Another embodiment described herein is a method for detecting one or more cardiac-associated microRNAs (miRNAs), the method comprising: obtaining a biological sample from a subject; extracting from the biological sample a liquid component comprising exosomes and miRNA from the heterogeneous biospecimen; subjecting the liquid component to an alternating current to lyse the exosomes and release exosomal miRNAs; concentrating the miRNAs proximate to an anion exchange membrane (AEM) functionalized with oligoprobes complementary to the miRNAs using a positively charged reservoir; generating a current-voltage curve (CVC) of the AEM and calculating the voltage shift from a CVC of the AEM absent miRNAs; quantifying the miRNAs bound to the oligoprobes using a calibration curve. In one aspect, the cardiac-associated miRNAs are selected from one or more of miR-1-1 (SEQ ID NO: 1); miR-208b (SEQ ID NO: 4); miR-499 (SEQ ID NO: 7); miR-200b (SEQ ID NO: 10); miR-543 (SEQ ID NO: 13); miR-331 (SEQ ID NO: 16); miR-3605 (SEQ ID NO: 19); miR-301a (SEQ ID NO: 22); miR-18a (SEQ ID NO: 25); miR-423 (SEQ ID NO: 28); miR-142 (SEQ ID NO: 31); or miR-132 (SEQ ID NO: 34). In another aspect, the miRNA is free, contained in exosomes, or a combination thereof. In another aspect, the calibration curve is generated by inputting the voltage shift (V) measured from the current-voltage curve (CVC) into the equation:

$$\frac{V}{RT / F} = A {log}_{10} ( \frac{C}{{C}_{r}} ) ,$$

wherein V is the voltage shift; R is the gas constant, 8.314 J (mol⁻¹·K⁻¹); T is the temperature, 25° C.=298 K; F is Faraday's constant, 9.648×10⁴ C mol⁻¹; A is a coefficient that is approximately the theoretical value of 2 ln₁₀(RT/F)≈0.12 V for every 10-fold decrease in bulk miRNA concentration; Cᵣ is the reference concentration; and C is the concentration of the miRNA bound to the oligoprobe. In another aspect, the method further comprises determining the concentration of the miRNAs in the biological sample and the miRNA concentrations of a control or normal subject. In another aspect, when the concentration of the miRNA in the biological sample is increased by 10-100-fold as compared to the control or normal subject, the subject is administered a treatment.

Another embodiment described herein is a system for detection of microRNA (miRNA) in a biological sample from a subject, the system comprising: an integration board comprising: a piezoelectric substrate comprising: a first inlet, a first outlet, and a fluidic channel fluidly connecting the first inlet and the first outlet; interdigitated electrodes; and a transducer that applies alternating current to the interdigitated electrodes; a second inlet, a second outlet, and the fluidic channel fluidly connecting the first outlet, the second inlet, and the second outlet; one or more ion exchange arrays capable of detecting one or more distinct miRNAs, the one or more ion exchange arrays comprising: a first cation exchange membrane (CEM) positioned across the fluidic channel proximate to the second inlet; a second CEM positioned across the fluidic channel proximate to the second outlet; a positively charged reservoir fluidly connected to the fluidic channel by the first CEM; a negatively charged reservoir fluidly connected to the fluidic channel by the second CEM; an anion exchange membrane (AEM) functionalized with a plurality of oligoprobes specific for a plurality of distinct miRNAs, fluidly connected to the fluidic channel and positioned between the first CEM and the second CEM; at least 2 source electrodes adapted to apply current across the AEM; and at least 2 sense electrodes adapted to measure voltage across the AEM; a potentiostat; a binary coding and decoding circuit to control and modulate the detection sensing for each distinct miRNA; a plurality of probe selection switches; a power source; and a device for inducing fluid flow through the system. In one aspect, a calibration curve is generated by inputting the voltage shift (V) measured from the current-voltage curve (CVC) into the equation:

$$\frac{V}{RT / F} = A {log}_{10} ( \frac{C}{{C}_{r}} ) ,$$

wherein V is the voltage shift; R is the gas constant, 8.314 J (mol⁻¹·K⁻¹); T is the temperature, 25° C.=298 K; F is Faraday's constant, 9.648×10⁴ C mol⁻¹; A is a coefficient that is approximately the theoretical value of 2 ln₁₀(RT/F)≈0.12 V for every 10-fold decrease in bulk miRNA concentration; Cᵣ is the reference concentration; and C is the concentration of the miRNA bound to the oligoprobe. In another aspect, the miRNA is one or more of miR-1-1 (SEQ ID NO: 1); miR-208b (SEQ ID NO: 4); miR-499 (SEQ ID NO: 7); miR-200b (SEQ ID NO: 10); miR-543 (SEQ ID NO: 13); miR-331 (SEQ ID NO: 16); miR-3605 (SEQ ID NO: 19); miR-301a (SEQ ID NO: 22); miR-18a (SEQ ID NO: 25); miR-423 (SEQ ID NO: 28); miR-142 (SEQ ID NO: 31); or miR-132 (SEQ ID NO: 34). In another aspect, the miRNA is free, contained in exosomes, or a combination thereof. In another aspect, the system is used to determine the concentration of the miRNAs in the biological sample and the miRNA concentrations of a control or normal subject. In another aspect, when the concentration of the miRNA in the biological sample is increased by 10-100-fold as compared to the control or normal subject, the subject is administered a treatment. In another aspect, the system does not require miRNA isolation, reverse-transcription, or preamplification. In another aspect, the system detects the miRNA in from about 30 minutes to about 45 minutes. In another aspect, the system detects at least two miRNAs simultaneously. In another aspect, when the miRNA is contained in exosomes, the system lyses the exosomes in about 1 minute. In another aspect, the lysis is mechanical lysis. In another aspect, the volume of the biological sample is from about 20 μL to about 40 μL.

Another embodiment described herein is a system for simultaneous detection of multiple distinct biomolecules in a biological sample from a subject, the system comprising: an integration board comprising: an inlet, an outlet, and a fluidic channel fluidly connecting the inlet and the outlet; one or more ion exchange arrays capable of detecting one or more distinct biomolecules, the one or more ion exchange arrays comprising: a first ion exchange membrane (IEM) positioned across the fluidic channel proximate to the inlet; a second IEM positioned across the fluidic channel proximate to the outlet; a first charged reservoir fluidly connected to the fluidic channel by the first IEM; a second charged reservoir fluidly connected to the fluidic channel by the second IEM; a third IEM functionalized with a plurality of probes specific for a plurality of distinct biomolecules, fluidly connected to the fluidic channel and positioned between the first IEM and the second IEM; a plurality of source electrodes adapted to apply current across the third IEM; and a plurality of sense electrodes adapted to measure voltage across the third IEM; a potentiostat; a binary coding and decoding circuit to control and modulate the detection sensing for each of the distinct biomolecules; a plurality of probe selection switches; a power source; and a device for inducing fluid flow through the system. In one aspect, the multiple distinct biomolecules are selected from nucleic acids, proteins, carbohydrates, lipids, or combinations thereof. In another aspect, a calibration curve is generated by inputting the voltage shift (V) measured from a current-voltage curve (CVC) into the equation:

$$\frac{V}{RT / F} = A {log}_{10} ( \frac{C}{{C}_{r}} ) ,$$

wherein V is the voltage shift; R is the gas constant, 8.314 J (mol⁻¹·K⁻¹); T is the temperature, 25° C.=298 K; F is Faraday's constant, 9.648×10⁴ C mol⁻¹; A is a coefficient that is approximately the theoretical value of 2 ln₁₀(RT/F)≈0.12 V for every 10-fold decrease in bulk concentration for each of the distinct biomolecules; Cᵣ is the reference concentration; and C is the concentration for each of the distinct biomolecules bound to the probe.

### DESCRIPTION OF THE DRAWINGS

The patent or application file contains at least one drawing executed in color. Copies of this patent or patent application publication with color drawing(s) will be provided by the Office upon request and payment of the necessary fee.

FIG. 1A-E show an illustration of the integrated measurement of miRNAs using a 3-membrane sensor. FIG. 1A shows the integration board including the detection device, the surface acoustic wave (SAW) lysing device, and the digital controlling. FIG. 1B shows the mechanism of the SAW lysing device breaking exosomes to release miRNAs. FIG. 1C shows the detection device including the MIX⋅miR sensors, a pre-concentration unit, and the electrodes connected to the potentiostat for CVC measurements. FIG. 1D shows the baseline measurement with three different kinds of ssDNA probes. FIG. 1E shows the measurement of miR-1 (SEQ ID NO: 1), miR-208b (SEQ ID NO: 4), and miR-499 (SEQ ID NO: 7).

FIG. 2A-J show the functionalization of the 3-membrane sensor with miR-1, miR-208b, and miR-499 and the CVC measurement procedure of each specific miRNA. FIG. 2A shows an illustration and a picture of the anion exchange membrane (AEM) embedded in the MIX⋅miR sensor. FIG. 2B shows the AEM carboxylation step using BPDA. FIG. 2C shows the attachment of 10 μM miRNA complement probes onto the AEM surfaces. FIG. 2D shows the MIX⋅miR sensor with three different kinds of miRNA complement probes attached. FIG. 2E shows a graph with the baseline measurement for the MIX⋅miR sensor with only the miRNA complements. FIG. 2F shows the miRNA sample during the pre-concentration step. FIG. 2G shows the high ionic wash and low ionic wash steps that remove non-specific miRNAs. FIG. 2H-J show three charts of the CVC curves for miR-1 (SEQ ID NO: 1), miR-208b (SEQ ID NO: 4), and miR-499 (SEQ ID NO: 7), respectively.

FIG. 3A-C show an example of an experimental setup. The electrode assignment in the CVC measurements is shown in FIG. 3A. The binary code selection of the electrodes for potentiostat wires is shown in Table 2, below. The integration board with fluidic devices and SAW lysing modulus during measurements is shown in FIG. 3B. The binary coding selection of different miRNAs with LED indication is shown in FIG. 3C.

FIG. 4A-C show the calibration curves of miR-1 (FIG. 4A, SEQ ID NO: 1), miR-208b (FIG. 4B; SEQ ID NO: 4), and miR-499 (FIG. 4C; SEQ ID NO: 7) using the standard concentrations of 0.1 pM, 1 pM, 10 pM, 100 pM, 1 nM, and 10 nM.

FIG. 5A-D show the concentrations of miR-1 (SEQ ID NO: 1), miR-208b (SEQ ID NO: 4), and miR-499 (SEQ ID NO: 7) in clinical samples with and without SAW lysis. FIG. 5A-B show the concentrations of the miRs with SAW lysis, and FIG. 5C-D show the concentrations of the miRs without SAW lysis. The error bars in FIG. 5B and FIG. 5D represent the 95% confidence level. LOD indicates the detection limit of the MIX.miR sensors. *The STEMI-PCI sample with SAW lysis is diluted by 5 times in 1×PBS. The results of the purple bar graphs with SAW lysing are multiplied by 5 after obtaining the concentrations).

FIG. 6A-B show miRNA isolation efficiency in PCR. FIG. 6A shows the isolation efficiency of different concentrations of miRNA. FIG. 6B shows the isolation concentrations (high, medium, low) compared with the miRNA measurement by the MIX.miR sensors.

FIG. 7 shows miR-1 PCR results of clinical samples: NCAD, CAD, STEMI-pre, and STEMI-PCI. The error bars represent one standard deviation.

FIG. 8A-E show the layout designs for exemplary integration boards. FIG. 8A shows the schematic of the Version 1 board. FIG. 8B-8D shows schematic of the Version 3 board. FIG. 8E shows a photograph of the Version 3 board.

FIG. 9 shows the voltage shifts for miR-200b (SEQ ID NO: 10); miR-543 (SEQ ID NO: 13); miR-331 (SEQ ID NO: 16); miR-3605 (SEQ ID NO: 19); miR-301a (SEQ ID NO: 22); miR-18a (SEQ ID NO: 25); miR-423 (SEQ ID NO: 28); miR-142 (SEQ ID NO: 31); and miR-132 (SEQ ID NO: 34).

FIG. 10A-E show radar maps illustrating the average voltage readings of 9 miRNAs, miR-200b (SEQ ID NO: 10); miR-543 (SEQ ID NO: 13); miR-331 (SEQ ID NO: 16); miR-3605 (SEQ ID NO: 19); miR-301a (SEQ ID NO: 22); miR-18a (SEQ ID NO: 25); miR-423 (SEQ ID NO: 28); miR-142 (SEQ ID NO: 31); and miR-132 (SEQ ID NO: 34) in 4 categories (all shown in FIG. 10A): NR (blue; FIG. 10B), CAD (green; FIG. 10C), STEMI-pre (red; FIG. 10D), and STEMI-PCI (magenta; FIG. 10E).

### DETAILED DESCRIPTION

Unless otherwise defined, all technical and scientific terms used herein have the same meaning as commonly understood by one of ordinary skill in the art. For example, any nomenclatures used in connection with, and techniques of, cell and tissue culture, molecular biology, immunology, microbiology, genetics, and protein and nucleic acid chemistry and hybridization described herein are well known and commonly used in the art. In case of conflict, the present disclosure, including definitions, will control. Exemplary methods and materials are described below, although methods and materials similar or equivalent to those described herein can be used in practice or testing of the embodiments and aspects described herein.

As used herein, the terms “amino acid,” “nucleotide,” “polynucleotide,” “vector,” “polypeptide,” and “protein” have their common meanings as would be understood by a biochemist of ordinary skill in the art. Standard single letter nucleotides (A, C, G, T, U) and standard single letter amino acids (A, C, D, E, F, G, H, I, K, L, M, N, P, Q, R, S, T, V, W, or Y) are used herein.

As used herein, the terms such as “include,” “including,” “contain,” “containing,” “having,” and the like mean “comprising.” The present disclosure also contemplates other embodiments “comprising,” “consisting of,” and “consisting essentially of,” the embodiments or elements presented herein, whether explicitly set forth or not.

As used herein, the term “a,” “an,” “the” and similar terms used in the context of the disclosure (especially in the context of the claims) are to be construed to cover both the singular and plural unless otherwise indicated herein or clearly contradicted by the context. In addition, “a,” “an,” or “the” means “one or more” unless otherwise specified.

As used herein, the term “or” can be conjunctive or disjunctive.

As used herein, the term “substantially” means to a great or significant extent, but not completely.

As used herein, the term “about” or “approximately” as applied to one or more values of interest, refers to a value that is similar to a stated reference value, or within an acceptable error range for the particular value as determined by one of ordinary skill in the art, which will depend in part on how the value is measured or determined, such as the limitations of the measurement system. In one aspect, the term “about” refers to any values, including both integers and fractional components that are within a variation of up to ±10% of the value modified by the term “about.” Alternatively, “about” can mean within 3 or more standard deviations, per the practice in the art. Alternatively, such as with respect to biological systems or processes, the term “about” can mean within an order of magnitude, in some embodiments within 5-fold, and in some embodiments within 2-fold, of a value. As used herein, the symbol “˜” means “about” or “approximately.”

All ranges disclosed herein include both end points as discrete values as well as all integers and fractions specified within the range. For example, a range of 0.1-2.0 includes 0.1, 0.2, 0.3, 0.4 . . . 2.0. If the end points are modified by the term “about,” the range specified is expanded by a variation of up to ±10% of any value within the range or within 3 or more standard deviations, including the end points.

As used herein, the terms “active ingredient” or “active pharmaceutical ingredient” refer to a pharmaceutical agent, active ingredient, compound, or substance, compositions, or mixtures thereof, that provide a pharmacological, often beneficial, effect.

As used herein, the terms “control,” or “reference” are used herein interchangeably. A “reference” or “control” level may be a predetermined value or range, which is employed as a baseline or benchmark against which to assess a measured result. “Control” also refers to control experiments or control cells.

As used herein, the term “dose” denotes any form of an active ingredient formulation or composition, including cells, that contains an amount sufficient to initiate or produce a therapeutic effect with at least one or more administrations. “Formulation” and “composition” are used interchangeably herein.

As used herein, the term “prophylaxis” refers to preventing or reducing the progression of a disorder, either to a statistically significant degree or to a degree detectable by a person of ordinary skill in the art.

As used herein, the terms “effective amount” or “therapeutically effective amount,” refers to a substantially non-toxic, but sufficient amount of an action, agent, composition, or cell(s) being administered to a subject that will prevent, treat, or ameliorate to some extent one or more of the symptoms of the disease or condition being experienced or that the subject is susceptible to contracting. The result can be the reduction or alleviation of the signs, symptoms, or causes of a disease, or any other desired alteration of a biological system. An effective amount may be based on factors individual to each subject, including, but not limited to, the subject's age, size, type or extent of disease, stage of the disease, route of administration, the type or extent of supplemental therapy used, ongoing disease process, and type of treatment desired.

As used herein, the terms “subject” and “patient” are used interchangeably. As used herein, the term “subject” refers to an animal. Typically, the subject is a mammal. A subject also refers to primates (e.g., humans, male or female; infant, adolescent, or adult), non-human primates, rats, mice, rabbits, pigs, cows, sheep, goats, horses, dogs, cats, fish, birds, and the like. In one embodiment, the subject is a primate. In one embodiment, the subject is a human.

As used herein, a subject is “in need of treatment” if such subject would benefit biologically, medically, or in quality of life from such treatment. A subject in need of treatment does not necessarily present symptoms, particular in the case of preventative or prophylaxis treatments.

As used herein, the terms “inhibit,” “inhibition,” or “inhibiting” refer to the reduction or suppression of a given biological process, condition, symptom, disorder, or disease, or a significant decrease in the baseline activity of a biological activity or process.

As used herein, “treatment” or “treating” refers to prophylaxis of, preventing, suppressing, repressing, reversing, alleviating, ameliorating, or inhibiting the progress of biological process including a disorder or disease, or completely eliminating a disease. A treatment may be either performed in an acute or chronic way. The term “treatment” also refers to reducing the severity of a disease or symptoms associated with such disease prior to affliction with the disease. “Repressing” or “ameliorating” a disease, disorder, or the symptoms thereof involves administering a cell, composition, or compound described herein to a subject after clinical appearance of such disease, disorder, or its symptoms. “Prophylaxis of” or “preventing” a disease, disorder, or the symptoms thereof involves administering a cell, composition, or compound described herein to a subject prior to onset of the disease, disorder, or the symptoms thereof. “Suppressing” a disease or disorder involves administering a cell, composition, or compound described herein to a subject after induction of the disease or disorder thereof but before its clinical appearance or symptoms thereof have manifest.

As used herein, “acute myocardial infarction” or “AMI” refers to myocardial necrosis resulting from reduction of blood supply to a portion of the myocardium typically from an acute obstruction of a coronary artery.

As used herein, “coronary artery disease” refers to the buildup of plaque in the coronary arteries that causes a narrowing or blockage that could result in acute myocardial infarction.

As used herein, “reperfusion injury” or “ischemia-reperfusion injury” or “IRI” refers to tissue damage that occurs after blood supply is restored after a period of ischemia or hypoxia. The absence of oxygen and nutrients from blood during the ischemic period creates a condition in which the restoration of circulation results in inflammation and oxidative damage through the induction of oxidative stress upon restoration of normal blood supply.

MicroRNAs (miRNAs) are small (17-22 nucleotides) non-coding RNAs that regulate gene expression at a posttranscriptional level by targeting messenger RNAs (mRNAs). miRNAs play a critical role in homeostasis, and their dysregulation has been associated with many disease states including acute myocardial infarction (AMI) and coronary artery disease (CAD). Links have been made to miRNA profiles in diagnostic, prognostic, and even therapeutic roles. Circulating miRNAs have recently been proposed as potential biomarkers for rapid and accurate assessment of several diseases such as cancer, AMI, CAD, and other cardiovascular diseases. As miRNA turnover is much quicker than that of proteins, miRNAs could additionally be used as a method to distinguish between AMI patients who have undergone reperfusion and those who have not, significantly decreasing the amount of testing needed prior to diagnosis.

Recent pre-clinical studies have shown several miRNAs, specifically miR-1-1 (SEQ ID NO: 1); miR-208b (SEQ ID NO: 4); miR-499 (SEQ ID NO: 7); miR-200b (SEQ ID NO: 10); miR-543 (SEQ ID NO: 13); miR-331 (SEQ ID NO: 16); miR-3605 (SEQ ID NO: 19); miR-301a (SEQ ID NO: 22); miR-18a (SEQ ID NO: 25); miR-423 (SEQ ID NO: 28); miR-142 (SEQ ID NO: 31); miR-132 (SEQ ID NO: 34) have the potential to accurately diagnose AMI. However, current methodology to detect changes in plasma miRNA levels relies on a series of procedures including quantitative reverse transcription PCR (RT-qPCR) analysis, which render the process lengthy and quantitatively unreliable, so that profiling of miRNAs is impractical to guide therapy in the setting of AMI. Specifically, plasma contains many PCR inhibitors and hence the miRNAs need to be extracted with a low-yield (<10%) extraction kit. The yield of this extraction pretreatment unit is further reduced by the lysing agents added to lyse the exosome carriers of the miRNAs. The conversion of 20-25 nucleotide miRNA to cDNA long enough for the primers involves a hairpin or toehold ligation step with a pairing sequence no longer than 10 bases. Such a short pairing sequence produces highly biased conversion that renders the quantification inaccurate. Extensive normalization is thus required for each miRNA, and it remains unclear if such normalization would be valid for all samples. An accurate pretreatment and RT-PCR free profiling technology is hence needed for miRNA profiling to be a viable diagnostic platform.

Microfluidic preconcentration/sensing technologies have shown promise in replacing qRT-PCR as an alternative for miRNA profiling. Electrochemical impedance spectroscopy (EIS), for example, can yield highly accurate quantification. However, because of spurious reactions with other reagents in the plasma sample, it is selectivity often undesirable unless tedious blocking of the electrode with surfactant monolayers is carried out. A new type of electrokinetic sensor based on ion-exchange polymer membranes (anion exchange membranes AEM for miRNA) eliminates this selectivity issue with a highly charged membrane surface that reduces attractive hydrophobic interaction with fouling plasma proteins. In the presence of a strong microfluidic wash flow, only target miRNAs that hybridize with their complementary oligonucleotides functionalized to the membrane surface will remain on the surface. Due to the ion depleting action of the membrane on the side of the functionalized oligonucleotide, the conductivity near that surface membrane is orders of magnitude lower than the bulk or within the charged membrane. The surface layer hence controls the voltage drop and the additional surface charge of the hybridized duplex can sensitively gate the ion current, producing a voltage signal much larger than those from electrochemical sensors. Additional ion-depleting membrane modules around the sensor can push and concentrate the charged analyte into a band around the sensor, thus increasing the local analytic concentration by as much as 3 orders of magnitude. When combined with a Surface Acoustic Wave (SAW) exosome lysing module that does not require the addition of diluting and contaminating lysing solutions, this membrane-based microfluidic platform has demonstrated high-sensitivity miRNA detection from plasma samples without pretreatment or PCR amplification. The same technology has been extended to low viral infectious disease detection that requires PCR amplification and to protein biomarker detection. The depletion membrane can also be used for high-yield isolation of the virus RNA from PCR inhibitors and nanoparticle reporters can be used for a sandwich assay of proteins.

One important issue concerning miRNA profiling is whether the miRNAs are exosomal. There is growing evidence that smaller nanocarriers like lipoproteins and ribonucleoproteins can also carry miRNAs, sometimes known as free-floating miRNAs. In earlier studies, evidence was found of miR21 in ribonucleoproteins when plasma from cancer patients is not lysed to release the exosomal miRNAs. However, gold standard methods for exosome isolation like ultra-centrifugation and size-exclusion chromatography are time consuming and low yield. The SAW lysing module permits the determination if the miRNAs are exosomal, as any incremental miRNAs after lysing must come from the exosomes. It allows for the determination if SAW lysing is necessary for the actual sample.

These early membrane platforms for miRNA quantify only one target and is hence sensitive to sample-to-sample variations because of the small volume used for the chip. The preconcentration module also has large variation in its concentration efficiency. Hence, miRNA profiling would be much more accurate if multiple miRNA targets in the concentrated band are quantified, with one of them serving as an invariant reference. Described herein is such a miRNA platform (MIX.miR: multiple-target ion-exchange-membrane-based miRNA), developed by integrating three specific miRNA AEM membrane sensors into a single capillary sensing unit and by combining this sensing capillary with membrane preconcentration and SAW-lysing modules. This platform is used to identify a panel of target miRNAs within clinical samples derived from four categories of clinical samples for AMI diagnosis.

Three candidate miRNAs closely associated with AMI, miR-1 (SEQ ID NO: 1), miR-208b (SEQ ID NO: 4), and miR-499 (SEQ ID NO: 7), were investigated from clinical plasma samples of (1) reference subjects with no evident coronary artery disease (NCAD); (2) subjects with stable coronary artery disease (CAD); and (3) subjects experiencing ST-elevation myocardial infarction (STEMI) prior to (STEMI-pre) and following percutaneous coronary intervention (STEMI-PCI). Additionally, all samples were tested with and without the integrated SAW lysis device to confirm whether the measured miRNA was free-floating or inside extracellular vesicles (e.g., exosomal). Measurements were then compared to the current gold standard detection of miRNA RT-qPCR measurements of the same samples.

The results from the diseased samples from this study match well with current literature for the miRNAs tested. In addition, for the first time, these results demonstrate the ability to distinguish between STEMI patients that have undergone clinical intervention resulting in reperfusion from those that have not, for each of miR-1 (SEQ ID NO: 1) and miR-208b (SEQ ID NO: 4), which suggests that these markers can distinguish between ongoing ischemia from reperfusion injury. These results also demonstrate the potential of the MIX.miR method as a low-cost point-of-care AMI diagnostic device providing results for multiple biomarkers in a more accurate and timely manner than current biomarker detection methods. This, along with its portability also makes the MIX.miR sensing platform a more viable AMI diagnostic tool in developing countries. Additionally, the MIX.miR can be utilized as a general miRNA quantification method that requires less sample preparation, is more accurate, and provides more rapid results than the current standard miRNA RT-qPCR.

One embodiment described herein is a method for detecting microRNAs (miRNAs) associated with acute myocardial infarction reperfusion injury, or coronary artery disease, the method comprising: obtaining a biological sample from a subject; and performing an assay on the biological sample to detect one or more miRNAs selected from miR-1-1 (SEQ ID NO: 1); miR-208b (SEQ ID NO: 4); miR-499 (SEQ ID NO: 7); miR-200b (SEQ ID NO: 10); miR-543 (SEQ ID NO: 13); miR-331 (SEQ ID NO: 16); miR-3605 (SEQ ID NO: 19); miR-301a (SEQ ID NO: 22); miR-18a (SEQ ID NO: 25); miR-423 (SEQ ID NO: 28); miR-142 (SEQ ID NO: 31); or miR-132 (SEQ ID NO: 34) by hybridization with oligonucleotide probes complementary to the one or more miRNAs. In one aspect, the method can distinguish between acute myocardial infarction, reperfusion injury, coronary artery disease, or normal subjects. In another aspect, the biological sample is selected from one or more of blood, serum, or plasma. In another aspect, the miRNA is free, contained in exosomes, or a combination thereof. In another aspect, the method further comprises determining the concentration of the miRNAs in the biological sample and the miRNA concentrations of a control or normal subject. In another aspect, when the concentration of the miRNA in the biological sample is increased by 10-100-fold as compared to the control or normal subject, the subject is administered a treatment.

Another embodiment described herein is a method for differentiation between acute myocardial infarction reperfusion injury, or coronary artery disease, the method comprising: obtaining a biological sample from a subject; and performing an assay on the biological sample to detect one or more miRNAs selected from miR-1-1 (SEQ ID NO: 1); miR-208b (SEQ ID NO: 4); miR-499 (SEQ ID NO: 7); miR-200b (SEQ ID NO: 10); miR-543 (SEQ ID NO: 13); miR-331 (SEQ ID NO: 16); miR-3605 (SEQ ID NO: 19); miR-301a (SEQ ID NO: 22); miR-18a (SEQ ID NO: 25); miR-423 (SEQ ID NO: 28); miR-142 (SEQ ID NO: 31); or miR-132 (SEQ ID NO: 34) by hybridization with oligonucleotide probes complementary to the one or more miRNAs. In one aspect, the biological sample is selected from one or more of blood, serum, or plasma. In another aspect, the miRNA is free, contained in exosomes, or a combination thereof. In another aspect, the method further comprises determining the concentration of the miRNAs in the biological sample and the miRNA concentrations of a control or normal subject. In another aspect, when the concentration of the miRNA in the biological sample is increased by 10-100-fold as compared to the control or normal subject, the subject is administered a treatment. In another aspect, the treatment comprises administering reperfusion therapy or a therapeutic selected from antiplatelet drugs, anticoagulants, nitrates, beta-blockers, statins, angiotensin-converting enzyme (ACE) inhibitors, angiotensin II receptor blockers (ARBs), calcium channel blockers, therapeutic hypothermia, free radical scavenger drugs, antioxidant drugs or vitamins, anesthesia, bile pigments, hydrogen (H₂), hydrogen sulfide (H₂S), nitrous oxide (NO), carbon monoxide (CO), or a combination thereof.

Another embodiment described herein is a method for detecting one or more cardiac-associated microRNAs (miRNAs), the method comprising: obtaining a biological sample from a subject; extracting from the biological sample a liquid component comprising exosomes and miRNA from the heterogeneous biospecimen; subjecting the liquid component to an alternating current to lyse the exosomes and release exosomal miRNAs; concentrating the miRNAs proximate to an anion exchange membrane (AEM) functionalized with oligoprobes complementary to the miRNAs using a positively charged reservoir, generating a current-voltage curve (CVC) of the AEM and calculating the voltage shift from a CVC of the AEM absent miRNAs; quantifying the miRNAs bound to the oligoprobes using a calibration curve. In one aspect, the cardiac-associated miRNAs are selected from one or more of miR-1-1 (SEQ ID NO: 1); miR-208b (SEQ ID NO: 4); miR-499 (SEQ ID NO: 7); miR-200b (SEQ ID NO: 10); miR-543 (SEQ ID NO: 13); miR-331 (SEQ ID NO: 16); miR-3605 (SEQ ID NO: 19); miR-301a (SEQ ID NO: 22); miR-18a (SEQ ID NO: 25); miR-423 (SEQ ID NO: 28); miR-142 (SEQ ID NO: 31); or miR-132 (SEQ ID NO: 34). In another aspect, the miRNA is free, contained in exosomes, or a combination thereof. In another aspect, the calibration curve is generated by inputting the voltage shift (V) measured from the current-voltage curve (CVC) into the equation:

$$\frac{V}{RT / F} = A {log}_{10} ( \frac{C}{{C}_{r}} ) ,$$

wherein V is the voltage shift; R is the gas constant, 8.314 J (mol⁻¹·K⁻¹); T is the temperature, 25° C.=298 K; F is Faraday's constant, 9.648×10⁴ C mol⁻¹; A is a coefficient that is approximately the theoretical value of 2 ln₁₀(RT/F)≈0.12 V for every 10-fold decrease in bulk miRNA concentration; Cᵣ is the reference concentration; and C is the concentration of the miRNA bound to the oligoprobe. In another aspect, the method further comprises determining the concentration of the miRNAs in the biological sample and the miRNA concentrations of a control or normal subject. In another aspect, when the concentration of the miRNA in the biological sample is increased by 10-100-fold as compared to the control or normal subject, the subject is administered a treatment.

Another embodiment described herein is a system for detection of microRNA (miRNA) in a biological sample from a subject, the system comprising: an integration board comprising: a piezoelectric substrate comprising: a first inlet, a first outlet, and a fluidic channel fluidly connecting the first inlet and the first outlet; interdigitated electrodes; and a transducer that applies alternating current to the interdigitated electrodes; a second inlet, a second outlet, and the fluidic channel fluidly connecting the first outlet, the second inlet, and the second outlet; one or more ion exchange arrays capable of detecting one or more distinct miRNAs, the one or more ion exchange arrays comprising: a first cation exchange membrane (CEM) positioned across the fluidic channel proximate to the second inlet; a second CEM positioned across the fluidic channel proximate to the second outlet; a positively charged reservoir fluidly connected to the fluidic channel by the first CEM; a negatively charged reservoir fluidly connected to the fluidic channel by the second CEM; an anion exchange membrane (AEM) functionalized with a plurality of oligoprobes specific for a plurality of distinct miRNAs, fluidly connected to the fluidic channel and positioned between the first CEM and the second CEM; at least 2 source electrodes adapted to apply current across the AEM; and at least 2 sense electrodes adapted to measure voltage across the AEM; a potentiostat; a binary coding and decoding circuit to control and modulate the detection sensing for each distinct miRNA; a plurality of probe selection switches; a power source; and a device for inducing fluid flow through the system. In one aspect, a calibration curve is generated by inputting the voltage shift (V) measured from the current-voltage curve (CVC) into the equation:

$$\frac{V}{RT / F} = A {log}_{10} ( \frac{C}{{C}_{r}} ) ,$$

wherein V is the voltage shift; R is the gas constant, 8.314 J (mol⁻¹K⁻¹); T is the temperature, 25° C.=298 K; F is Faraday's constant, 9.648×10⁴ C mol⁻¹; A is a coefficient that is approximately the theoretical value of 2 ln₁₀(RT/F)≈0.12 V for every 10-fold decrease in bulk miRNA concentration; Cᵣ is the reference concentration; and C is the concentration of the miRNA bound to the oligoprobe. In another aspect, the miRNA is one or more of miR-1-1 (SEQ ID NO: 1); miR-208b (SEQ ID NO: 4); miR-499 (SEQ ID NO: 7); miR-200b (SEQ ID NO: 10); miR-543 (SEQ ID NO: 13); miR-331 (SEQ ID NO: 16); miR-3605 (SEQ ID NO: 19); miR-301a (SEQ ID NO: 22); miR-18a (SEQ ID NO: 25); miR-423 (SEQ ID NO: 28); miR-142 (SEQ ID NO: 31); or miR-132 (SEQ ID NO: 34). In another aspect, the miRNA is free, contained in exosomes, or a combination thereof. In another aspect, the system is used to determine the concentration of the miRNAs in the biological sample and the miRNA concentrations of a control or normal subject. In another aspect, when the concentration of the miRNA in the biological sample is increased by 10-100-fold as compared to the control or normal subject, the subject is administered a treatment. In another aspect, the system does not require miRNA isolation, reverse-transcription, or preamplification. In another aspect, the system detects the miRNA in from about 30 minutes to about 45 minutes. In another aspect, the system detects at least two miRNAs simultaneously. In another aspect, when the miRNA is contained in exosomes, the system lyses the exosomes in about 1 minute. In another aspect, the lysis is mechanical lysis. In another aspect, the volume of the biological sample is from about 20 μL to about 40 μL.

Another embodiment described herein is a system for simultaneous detection of multiple distinct biomolecules in a biological sample from a subject, the system comprising: an integration board comprising: an inlet, an outlet, and a fluidic channel fluidly connecting the inlet and the outlet; one or more ion exchange arrays capable of detecting one or more distinct biomolecules, the one or more ion exchange arrays comprising: a first ion exchange membrane (IEM) positioned across the fluidic channel proximate to the inlet; a second IEM positioned across the fluidic channel proximate to the outlet; a first charged reservoir fluidly connected to the fluidic channel by the first IEM; a second charged reservoir fluidly connected to the fluidic channel by the second IEM; a third IEM functionalized with a plurality of probes specific for a plurality of distinct biomolecules, fluidly connected to the fluidic channel and positioned between the first IEM and the second IEM; a plurality of source electrodes adapted to apply current across the third IEM; and a plurality of sense electrodes adapted to measure voltage across the third IEM; a potentiostat; a binary coding and decoding circuit to control and modulate the detection sensing for each of the distinct biomolecules; a plurality of probe selection switches; a power source; and a device for inducing fluid flow through the system. In one aspect, the multiple distinct biomolecules are selected from nucleic acids, proteins, carbohydrates, lipids, or combinations thereof. In another aspect, a calibration curve is generated by inputting the voltage shift (V) measured from a current-voltage curve (CVC) into the equation:

$$\frac{V}{RT / F} = A {log}_{10} ( \frac{C}{{C}_{r}} ) ,$$

wherein V is the voltage shift; R is the gas constant, 8.314 J (mol⁻¹·K⁻¹); T is the temperature, 25° C.=298 K; F is Faraday's constant, 9.648×10⁴ C mol⁻¹; A is a coefficient that is approximately the theoretical value of 2 ln₁₀(RT/F)≈0.12 V for every 10-fold decrease in bulk concentration for each of the distinct biomolecules; Cᵣ is the reference concentration; and C is the concentration for each of the distinct biomolecules bound to the probe.

It will be apparent to one of ordinary skill in the relevant art that suitable modifications and adaptations to the compositions, formulations, methods, processes, and applications described herein can be made without departing from the scope of any embodiments or aspects thereof. The compositions and methods provided are exemplary and are not intended to limit the scope of any of the specified embodiments. All of the various embodiments, aspects, and options disclosed herein can be combined in any variations or iterations. The scope of the compositions, formulations, methods, and processes described herein include all actual or potential combinations of embodiments, aspects, options, examples, and preferences herein described.

The exemplary compositions and formulations described herein may omit any component, substitute any component disclosed herein, or include any component disclosed elsewhere herein. The ratios of the mass of any component of any of the compositions or formulations disclosed herein to the mass of any other component in the formulation or to the total mass of the other components in the formulation are hereby disclosed as if they were expressly disclosed. Should the meaning of any terms in any of the patents or publications incorporated by reference conflict with the meaning of the terms used in this disclosure, the meanings of the terms or phrases in this disclosure are controlling. Furthermore, the foregoing discussion discloses and describes merely exemplary embodiments. All patents and publications cited herein are incorporated by reference herein for the specific teachings thereof.

Various embodiments and aspects of the inventions described herein are summarized by the following clauses:
Clause 1. A method for detecting microRNAs (miRNAs) associated with acute myocardial infarction reperfusion injury, or coronary artery disease, the method comprising:
   obtaining a biological sample from a subject; and
   performing an assay on the biological sample to detect one or more miRNAs selected from miR-1-1 (SEQ ID NO: 1); miR-208b (SEQ ID NO: 4); miR-499 (SEQ ID NO: 7); miR-200b (SEQ ID NO: 10); miR-543 (SEQ ID NO: 13); miR-331 (SEQ ID NO: 16); miR-3605 (SEQ ID NO: 19); miR-301a (SEQ ID NO: 22); miR-18a (SEQ ID NO: 25); miR-423 (SEQ ID NO: 28); miR-142 (SEQ ID NO: 31); or miR-132 (SEQ ID NO: 34) by hybridization with oligonucleotide probes complementary to the one or more miRNAs.
Clause 2. The method of clause 1, wherein the method can distinguish between acute myocardial infarction, reperfusion injury, coronary artery disease, or normal subjects.
Clause 3. The method of clause 1 or 2, wherein the biological sample is selected from one or more of blood, serum, or plasma.
Clause 4. The method of any one of clauses 1-3, wherein the miRNA is free, contained in exosomes, or a combination thereof.
Clause 5. The method of any one of clauses 1-4, further comprising determining the concentration of the miRNAs in the biological sample and the miRNA concentrations of a control or normal subject.
Clause 6. The method of any one of clauses 1-5, wherein when the concentration of the miRNA in the biological sample is increased by 10-100-fold as compared to the control or normal subject, the subject is administered a treatment.
Clause 7. A method for differentiation between acute myocardial infarction reperfusion injury, or coronary artery disease, the method comprising:
   obtaining a biological sample from a subject; and
   performing an assay on the biological sample to detect one or more miRNAs selected from miR-1-1 (SEQ ID NO: 1); miR-208b (SEQ ID NO: 4); miR-499 (SEQ ID NO: 7); miR-200b (SEQ ID NO: 10); miR-543 (SEQ ID NO: 13); miR-331 (SEQ ID NO: 16); miR-3605 (SEQ ID NO: 19); miR-301a (SEQ ID NO: 22); miR-18a (SEQ ID NO: 25); miR-423 (SEQ ID NO: 28); miR-142 (SEQ ID NO: 31); or miR-132 (SEQ ID NO: 34) by hybridization with oligonucleotide probes complementary to the one or more miRNAs.
Clause 8. The method of clause 7, wherein the biological sample is selected from one or more of blood, serum, or plasma.
Clause 9. The method of clause 7 or 8, wherein the miRNA is free, contained in exosomes, or a combination thereof.
Clause 10. The method of any one of clauses 7-9, further comprising determining the concentration of the miRNAs in the biological sample and the miRNA concentrations of a control or normal subject.
Clause 11. The method of any one of clauses 7-10, wherein when the concentration of the miRNA in the biological sample is increased by 10-100-fold as compared to the control or normal subject, the subject is administered a treatment.
Clause 12. The method of any one of clauses 7-11, wherein the treatment comprises administering reperfusion therapy or a therapeutic selected from antiplatelet drugs, anticoagulants, nitrates, beta-blockers, statins, angiotensin-converting enzyme (ACE) inhibitors, angiotensin II receptor blockers (ARBs), calcium channel blockers, therapeutic hypothermia, free radical scavenger drugs, antioxidant drugs or vitamins, anesthesia, bile pigments, hydrogen (H₂), hydrogen sulfide (H₂S), nitrous oxide (NO), carbon monoxide (CO), or a combination thereof.
Clause 13. A method for detecting one or more cardiac-associated microRNAs (miRNAs), the method comprising:
   obtaining a biological sample from a subject;
   extracting from the biological sample a liquid component comprising exosomes and miRNA from the heterogeneous biospecimen;
   subjecting the liquid component to an alternating current to lyse the exosomes and release exosomal miRNAs;
   concentrating the miRNAs proximate to an anion exchange membrane (AEM) functionalized with oligoprobes complementary to the miRNAs using a positively charged reservoir;
   generating a current-voltage curve (CVC) of the AEM and calculating the voltage shift from a CVC of the AEM absent miRNAs;
   quantifying the miRNAs bound to the oligoprobes using a calibration curve.
Clause 14. The method of clause 13, wherein the cardiac-associated miRNAs are selected from one or more of miR-1-1 (SEQ ID NO: 1); miR-208b (SEQ ID NO: 4); miR-499 (SEQ ID NO: 7); miR-200b (SEQ ID NO: 10); miR-543 (SEQ ID NO: 13); miR-331 (SEQ ID NO: 16); miR-3605 (SEQ ID NO: 19); miR-301a (SEQ ID NO: 22); miR-18a (SEQ ID NO: 25); miR-423 (SEQ ID NO: 28); miR-142 (SEQ ID NO: 31); or miR-132 (SEQ ID NO: 34).
Clause 15. The method of clause 13 or 14, wherein the miRNA is free, contained in exosomes, or a combination thereof.
Clause 16. The method of any one of clauses 13-15, wherein the calibration curve is generated by inputting the voltage shift (V) measured from the current-voltage curve (CVC) into the equation:

$$\frac{V}{RT / F} = A {log}_{10} ( \frac{C}{{C}_{r}} ) ,$$

wherein V is the voltage shift; R is the gas constant, 8.314 J (mol⁻¹·K⁻¹); T is the temperature, 25° C.=298 K; F is Faraday's constant, 9.648×10⁴ C mol⁻¹; A is a coefficient that is approximately the theoretical value of 2 ln₁₀(RT/F)≈0.12 V for every 10-fold decrease in bulk miRNA concentration; Cᵣ is the reference concentration; and C is the concentration of the miRNA bound to the oligoprobe.
Clause 17. The method of any one of clauses 13-16, further comprising determining the concentration of the miRNAs in the biological sample and the miRNA concentrations of a control or normal subject.
Clause 18. The method of any one of clauses 13-17, wherein when the concentration of the miRNA in the biological sample is increased by 10-100-fold as compared to the control or normal subject, the subject is administered a treatment.
Clause 19. A system for detection of microRNA (miRNA) in a biological sample from a subject, the system comprising:
   an integration board comprising:
      a piezoelectric substrate comprising:
         a first inlet, a first outlet, and a fluidic channel fluidly connecting the first inlet and the first outlet;
         interdigitated electrodes; and
         a transducer that applies alternating current to the interdigitated electrodes;
      a second inlet, a second outlet, and the fluidic channel fluidly connecting the first outlet, the second inlet, and the second outlet;
      one or more ion exchange arrays capable of detecting one or more distinct miRNAs, the one or more ion exchange arrays comprising:
         a first cation exchange membrane (CEM) positioned across the fluidic channel proximate to the second inlet;
         a second CEM positioned across the fluidic channel proximate to the second outlet;
         a positively charged reservoir fluidly connected to the fluidic channel by the first CEM;
         a negatively charged reservoir fluidly connected to the fluidic channel by the second CEM;
         an anion exchange membrane (AEM) functionalized with a plurality of oligoprobes specific for a plurality of distinct miRNAs, fluidly connected to the fluidic channel and positioned between the first CEM and the second CEM;
         at least 2 source electrodes adapted to apply current across the AEM; and
         at least 2 sense electrodes adapted to measure voltage across the AEM;
      a potentiostat;
      a binary coding and decoding circuit to control and modulate the detection sensing for each distinct miRNA;
      a plurality of probe selection switches;
      a power source; and
   a device for inducing fluid flow through the system.
Clause 20. The system of clause 19, wherein a calibration curve is generated by inputting the voltage shift (V) measured from the current-voltage curve (CVC) into the equation:

$$\frac{V}{RT / F} = A {log}_{10} ( \frac{C}{{C}_{r}} ) ,$$

wherein V is the voltage shift; R is the gas constant, 8.314 J (mol⁻¹·K⁻¹); T is the temperature, 25° C.=298 K; F is Faraday's constant, 9.648×10⁴ C mol⁻¹; A is a coefficient that is approximately the theoretical value of 2 ln₁₀(RT/F)≈0.12 V for every 10-fold decrease in bulk miRNA concentration; Cᵣ is the reference concentration; and C is the concentration of the miRNA bound to the oligoprobe.
Clause 21. The system of clause 19 or 20, wherein the miRNA is one or more of miR-1-1 (SEQ ID NO: 1); miR-208b (SEQ ID NO: 4); miR-499 (SEQ ID NO: 7); miR-200b (SEQ ID NO: 10); miR-543 (SEQ ID NO: 13); miR-331 (SEQ ID NO: 16); miR-3605 (SEQ ID NO: 19); miR-301a (SEQ ID NO: 22); miR-18a (SEQ ID NO: 25); miR-423 (SEQ ID NO: 28); miR-142 (SEQ ID NO: 31); or miR-132 (SEQ ID NO: 34).
Clause 22. The system of any one of clauses 19-21, wherein the miRNA is free, contained in exosomes, or a combination thereof.
Clause 23. The system of any one of clauses 19-22, wherein the system is used to determine the concentration of the miRNAs in the biological sample and the miRNA concentrations of a control or normal subject.
Clause 24. The system of any one of clauses 19-23, wherein when the concentration of the miRNA in the biological sample is increased by 10-100-fold as compared to the control or normal subject, the subject is administered a treatment.
Clause 25. The system of any one of clauses 19-24, wherein the system does not require miRNA isolation, reverse-transcription, or preamplification.
Clause 26. The system of any one of clauses 19-25, wherein the system detects the miRNA in from about 30 minutes to about 45 minutes.
Clause 27. The system of any one of clauses 19-26, wherein the system detects at least two miRNAs simultaneously.
Clause 28. The system of any one of clauses 19-22, wherein when the miRNA is contained in exosomes, the system lyses the exosomes in about 1 minute.
Clause 29. The system of clause 28, wherein the lysis is mechanical lysis.
Clause 30. The system of any one of clauses 19-29, wherein the volume of the biological sample is from about 20 μL to about 40 μL.
Clause 31. A system for simultaneous detection of multiple distinct biomolecules in a biological sample from a subject, the system comprising:
   an integration board comprising:
      an inlet, an outlet, and a fluidic channel fluidly connecting the inlet and the outlet;
      one or more ion exchange arrays capable of detecting one or more distinct biomolecules, the one or more ion exchange arrays comprising:
         a first ion exchange membrane (IEM) positioned across the fluidic channel proximate to the inlet;
         a second IEM positioned across the fluidic channel proximate to the outlet;
         a first charged reservoir fluidly connected to the fluidic channel by the first IEM;
         a second charged reservoir fluidly connected to the fluidic channel by the second IEM;
         a third IEM functionalized with a plurality of probes specific for a plurality of distinct biomolecules, fluidly connected to the fluidic channel and positioned between the first IEM and the second IEM;
         a plurality of source electrodes adapted to apply current across the third IEM; and
         a plurality of sense electrodes adapted to measure voltage across the third IEM;
      a potentiostat;
      a binary coding and decoding circuit to control and modulate the detection sensing for each of the distinct biomolecules;
      a plurality of probe selection switches;
      a power source; and
   a device for inducing fluid flow through the system.
Clause 32. The system of claim 31, wherein the multiple distinct biomolecules are selected from nucleic acids, proteins, carbohydrates, lipids, or combinations thereof.
Clause 33. The system of claim 31 or 32, wherein a calibration curve is generated by inputting the voltage shift (V) measured from a current-voltage curve (CVC) into the equation:

$$\frac{V}{RT / F} = A {log}_{10} ( \frac{C}{{C}_{r}} ) ,$$

wherein V is the voltage shift; R is the gas constant, 8.314 J (mol⁻¹·K⁻¹); T is the temperature, 25° C.=298 K; F is Faraday's constant, 9.648×10⁴ C mol⁻¹; A is a coefficient that is approximately the theoretical value of 2 ln₁₀(RT/F)≈0.12 V for every 10-fold decrease in bulk concentration for each of the distinct biomolecules; Cᵣ is the reference concentration; and C is the concentration for each of the distinct biomolecules bound to the probe.

### EXAMPLES

### Example 1

### Sample Collection and Preparation

Blood was collected via standard venous puncture from healthy subjects, subjects diagnosed with CAD, and patients diagnosed with STEMI with an observed obstruction (STEMI-pre) and following reperfusion (STEMI-PCI) (6 patients for all sample groups) into tubes containing ethylenediaminetetraacetic acid (EDTA). After collection, plasma was isolated by centrifuging at 1000×g for 5 min and the plasma was transferred to RNA free tubes and stored at −80° C. The clinical samples were thawed on ice, aliquoted, and stored at −80° C. until testing with the MIX.miR sensors.

Oligoprobes and Calibration miRNAs

The oligoprobes and calibration miRNAs are purchased from Integrated DNA Technologies, Inc. The miRNAs, calibration oligonucleotides and capture oligonucleotides are shown in Table 1.

TABLE 1
miRNAs, Calibration Oligonucleotides, and Capture Oligonucleotide Sequences

| | | Calibration | Capture Oligonucleotide |
|---|---|---|---|
| miRNA | miRNA Sequence (5′→3′) | Oligonucleotide (5′→3′) | (5′→3′) |
| miR-1-1 | UGGAAUGUAAAGAAGUAUGUAU | TGGAATGTAAAGAAGTATGTAT | /SAmMC12/ATACATACTTCTTT |
| | (SEQ ID NO: 1) | (SEQ ID NO: 2) | ACATTCCA |
| | | | (SEQ ID NO: 3) |
| miR-208b | AUAAGACGAACAAAAGGUUUGU | ATAAGACGAACAAAAGGTTTGT | /5AmMC12/ACAAACCTTTTGTT |
| | (SEQ ID NO: 4) | (SEQ ID NO: 5) | GGTCTTAT |
| | | | (SEQ ID NO: 6) |
| miR-499- | UUAAGACUUGCAGUGAUGUUU | TTAAGACTTGCAGIGATGTTT | /5AmMC12/AAACATCACTGCAA |
| 5p | (SEQ ID NO: 7) | (SEQ ID NO: 8) | GTCTTAA |
| | | | (SEQ ID NO: 9) |
| ath-miR- | UUUGGAUUGAAGGGAGCUCUA | | |
| 159a | (SEQ ID NO: 37) | | |

The calibration oligonucleotides and capture oligonucleotides were aliquoted and stored at −20° C.

miRNA Isolation for RT-qPCR

miRNA was isolated from human plasma using the Maxwell RSC instrument (Promega, Wisconsin, USA) and miRNA plasma or serum miRNA isolation kit (Promega) with lysing solution and RNA paramagnetic bead cartridge extraction, as per the provided miRNA isolation protocol. Following miRNA isolation, samples were spiked with miRNA ath-miR-159a (SEQ ID NO: 37; Integrated DNA Technologies) at a concentration of 20 nM. cDNA was synthesized from the isolated miRNA for each specific miRNA that was to be tested: miR-1 (SEQ ID NO: 1), miR-208b (SEQ ID NO: 4), miR-499 (SEQ ID NO: 7), and ath-miR-159a (SEQ ID NO: 37); Thermo Fisher Scientific) using the custom Taqman reverse transcription for each miRNA following the provided Taqman custom reverse transcription protocol (Thermo Fisher). Preamplification was performed using Taqman Preamp Mastermix (Thermo Fisher) and custom miRNA assays for each miRNA tested miR-1 (SEQ ID NO: 1), miR-208b (SEQ ID NO: 4), miR-499 (SEQ ID NO: 7), and ath-miR-159a (SEQ ID NO: 37); Thermo Fisher) following the provided Taqman custom preamplification protocol for 16 cycles. miRNA RT-qPCR was performed using the Taqman custom miRNA assays for each specific miRNA tested miR-1 (SEQ ID NO: 1), miR-208b (SEQ ID NO: 4), miR-499 (SEQ ID NO: 7), and ath-miR-159a (SEQ ID NO: 37); Thermo Fisher) following the provided protocol. For data analysis, the delta-delta CT method was utilized using the spiked in ath-miR-159a (SEQ ID NO: 37) as the housekeeping miRNA.

miRNA Isolation Efficiency Assay for RT-qPCR

A known mass (0.05 pg, 5 pg, and 500 pg) of ath-miR-159a (SEQ ID NO: 37) was spiked into 300 μL of RNase free water at three different concentrations. miRNA was then isolated from the samples using the Qiagen miRNeasy kit (Qiagen) following the protocol as per manufacturer instructions. Following isolation, an additional clean up step was performed by running the miRNA solution through a 3 kDa filtration column (Sigma Aldrich) at 14,000×g for 90 min and extracting the purified miRNA, after which the volume was normalized to 25 μL. The same 3 amounts of ath-miR-159a (SEQ ID NO: 37) was also spiked into 25 μL of RNase free water as control. miRNA PCR was then performed as described above and expression of the isolated samples was normalized to the spiked in samples that did not undergo miRNA isolation.

### Example 2

### Integration Board

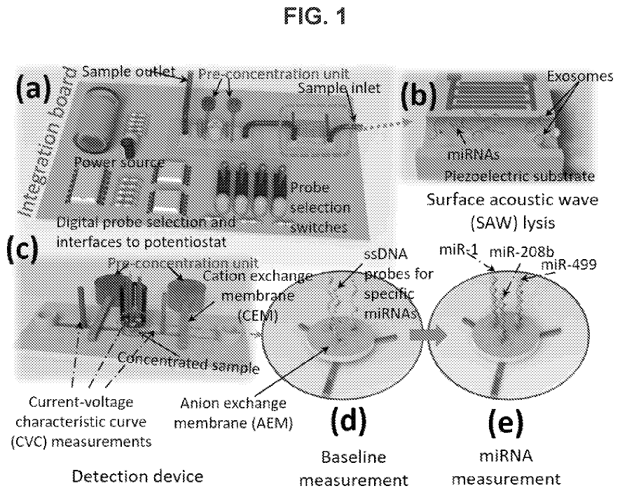

Fluidic device fabrication utilized previously established procedures. The sensing capillary with 3 anion-exchange membrane (AEM) sensors for 3 different miRNAs is similar to an earlier design for 4 serotypes of dengue RNA. The AEM contains nanoporous (<1 nm) ion-selective granules in the membrane, which allows ion depletion across the membrane with an onset voltage. As illustrated in FIG. 1, the miRNAs will bond with the specific oligoprobes. miR-1 (SEQ ID NO: 1), miR-208b (SEQ ID NO: 4), miR-499 (SEQ ID NO: 7) were used in the study with the clinical MI samples. Once the miRNAs attach to the AEM, the CVC (Current-Voltage Curve) of the AEM will generate a voltage shift compared to the baseline voltage of oligoprobes on the AEM.

Briefly, the samples were thawed on ice and a volume of 20 μL were lysed using the surface acoustic wave (SAW) lysing device. The SAW will break the extracellular vesicles such as exosomes, releasing their miRNAs into the sample. The sample is then flowed through the sensing channel of the MIX.miR sensor by a syringe pump at a constant flow rate of 250 μL/h. The flow channel is made of polycarbonate to reduce possible miRNA attraction with polydimethylsiloxane (PDMS). The AEM-based sensor is polyurethane for the same reason. The dimension of the flow channel is 45 mm in length, 300 μm in height, and 2 mm in width. An additional 200 mL of 1× phosphate-buffered saline (PBS) was used as driving fluid for the sensing channel. A pre-concentration unit, with two additional cation-exchange membranes (CEM), was activated with a constant current of 8×10⁻⁴ A to keep the miRNAs near the MIX.miR sensor for 20 min. The connection between the pre-concentration power and the fluidic channel is a pair of CEMs. Both the AEM and CEM belong to the family of ion-exchange membranes (IEM) with the only difference being the polarity of the opposite ion transfer orientation. After several washes by PBS, the CVC of the sensor was measured in 0.1×PBS by a potentiostat connected to the integration board. The probe selection switches on the board can manually select each pair of electrodes for the potentiostat.

The integration board contains a binary coding and decoding circuit to control the selection of each miRNA target. The SAW lysing device can break down exosomes in the plasma samples to release miRNAs. The SAW device was built on a piezoelectric substrate with interdigitated electrodes. The sinusoid signal for generating the SAW is 28.16 MHz with 190 mV Vpp (peak to peak voltage, Agilent 33250A, Agilent Technologies, Inc.). The signal was amplified to a power of 1 W via a RF power amplifier (Electronics & Innovation 325LA RF). To maintain the integrity of the miRNAs, ice chips were placed around the SAW device and sample inlet tubing. The SAW lysing device was washed by isopropanol (IPA) and de-ionized (DI) water after each usage.

### MIX.miR Sensor

The MIX.miR sensor was prepared by polyurethane (PU) molding with AEM embedded on the surface. The raw molds for the AEM sensor are printed by 3D wax printer (Solidscape Studio) with building wax as a sacrificial material. The molds for PU are made by mixing TAP silicone and catalyst (TAP Plastic) at a weight ratio of 10:1. The silicone mixture was cast on the wax mold. The wax mold was later dissolved in dimethyl sulfoxide (DMSO), followed by rinsing with IPA. The replica molding of TAP silicone was done following the regular PDMS casting methods with the assist of 3D wax printing to enhance the consistency of the MIX.miR sensors. The PU is solidified from the mixture of a two-component PU casting resin (TAP Plastic) at a weight ratio of 1:1. To covalently link the miRNA probes to the 3 AEMs, 20 μL of 60 μg/pL 3,3′,4,4′-benzophenonetetracarboxylic dianhydride (BPDA) was added to the AEM surface and exposed to UV light for 90 s. After the carboxylation procedures (modifying the AEM surface with —COOH groups), three different complementary miRNA probes, miR-1 complement (SEQ ID NO: 3), miR-208b complement (SEQ ID NO: 6), and miR-499 complement (SEQ ID NO: 9) which contain a 5′-amino-(CH₂)₁₂-phosphate modification, e.g., 5′-NH₂(CH₂)₁₂PO₄— (5AmMC₁₂; Integrated DNA Technologies, Inc.), were attached on each AEM using EDC/NHS coupling chemistry and incubated at 4° C. for 8 hours. Due to the presence of the additional Y-shaped barrier, the three different miRNAs cannot merge with each other, resulting in the functionalization of one probe to one AEM without any cross contamination.

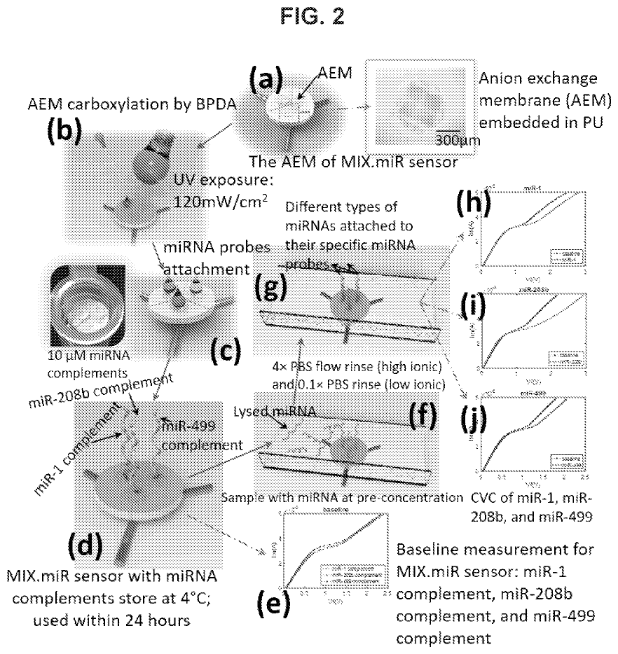

The baselines of the miRNA probes were measured first. The device went through a high ionic wash with 4×PBS to remove any miRNA complement and followed by a low ionic wash with 0.1×PBS to remove any high ionic residual. As illustrated in FIG. 2, the baselines for the 3 membranes showed linear regions, saturation regions, and over-limiting regions. As demonstrated in FIG. 2, only the miRNA complements attached to the AEM. The sample either with or without SAW lysing was pumped into the fluidic device during pre-concentration. The miRNAs were able to attach to their specific miRNA probes. After another high ionic wash with 4×PBS, the remaining miRNA statically adhering to the AEM were removed. Finally, multiple low ionic washes with 0.1×PBS were performed leaving only the targeted miRNAs covalently bonded to their specific oligoprobes. The CVC showed a voltage shift at the over-limiting region with twice the current in the saturation region.

Due to the depletion action by the AEM sensor and by an ensuing electroconvective instability, the CVC curve is nonlinear, with a linear Ohmic region, a saturated limiting-current region and a second linear over-limiting region with increasing voltage. The limiting current region results from the depletion action on the membrane surface, which increases the system resistance, and the over-limiting region is because ions are replenished into this depletion region by the electroconvective instability. As shown in FIG. 2, the baseline measurements of the three different membranes are almost overlapping in the linear regions and the over-limiting regions. This manufacturing method hence displayed promising consistency between the different AEM sensors. From the measurement of miR-1 (FIG. 2 inset red dots curve), miR-208b (FIG. 2 inset greed dots curve), and miR-499 FIG. 2 inset (blue dots curve), the voltage shifts in the over-limiting regions compared with the baselines (FIG. 2 inset black dots curves in all three charts) are indicating the different concentrations of each specific miRNA in the sample.

### Experimental Assay Setup

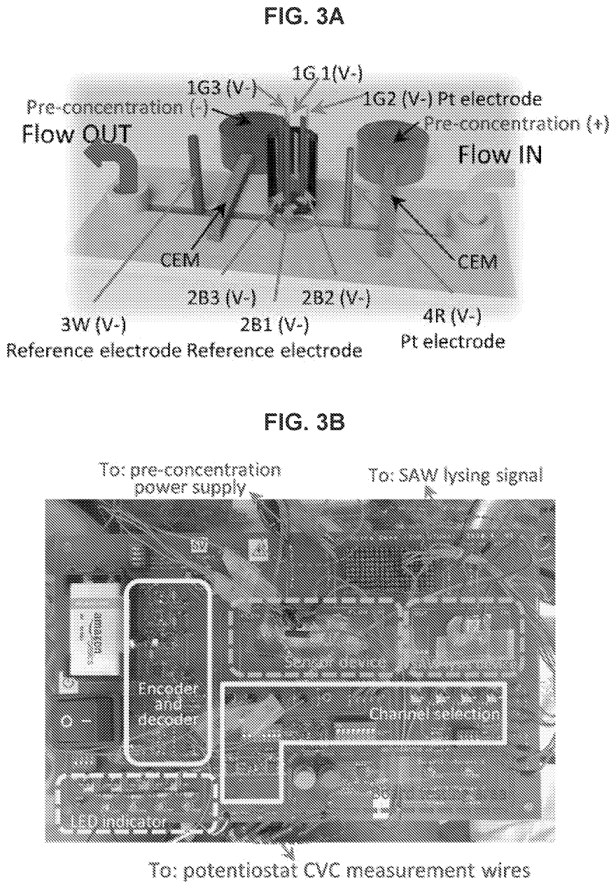

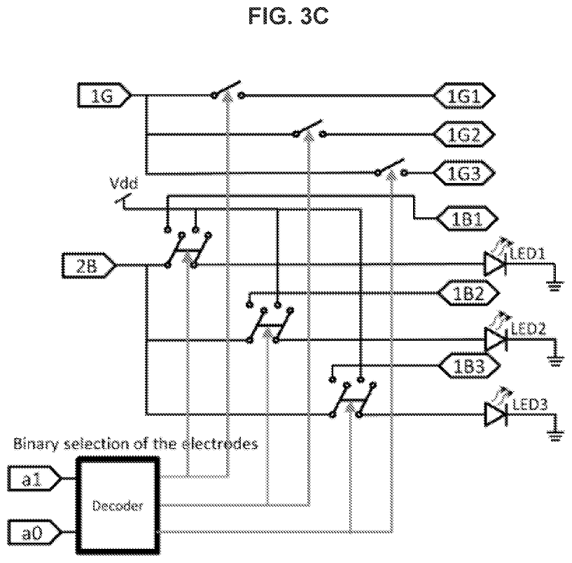

The CVC is measured by the potentiostat with 4 electrodes: 2 platinum electrodes providing current input, and 2 reference electrodes collecting the voltage across the AEM. After the miRNAs hybridized with the miRNA probes, the CVC will show an increase of the voltage at the over-limiting region of the AEM, at the same current, due to the enhancement of the electroconvective instability that replenishes ions into the depleted region near the membrane. As illustrated in FIG. 3A, the sensing electrodes are assigned with the color codes consistent with the wire color of the potentiostat (Gamry Reference 600, Gamry Instruments). The electrodes 1G (green) and 4R (red) are applying current across the AEM, while 2B (blue) and 3W (white) are measuring the voltage across the AEM. The current applying on 1G and 4R is 0-8×10⁻⁵ A, with a scanning step of 1 μA/s. Here, the three membrane chambers are sharing the common 3W and 4R. Table 2 shows the binary coding assignments of the electrodes for potentiostat wires shown in FIG. 3A: #1, Green; #2, Blue; #3, White; and #4, Red.

TABLE 2
Binary coding and the assignments of electrodes

| | I/O | | | |
|---|---|---|---|---|
| Code | 1G(I+) | 2B (V+) | 3W (V−) | 4R (I−) |
| a₁a₀ = 00 | 1G1 | 2B1 | com | com |
| a₁a₀ = 01 | 1G2 | 2B2 | com | com |
| a₁a₀ = 10 | 1G3 | 2B3 | com | com |
| a₁a₀ = 11 | NC | NC | NC | NC |

I/O: Input/output;
G: green;
B: Blue;
W: white;
R: Red;
com: common port;
NC: not connected.

The pre-concentration unit is used to keep the negatively charged miRNAs in the sample near the AEM sensors with oligoprobes. The pre-concentration units are made by two pieces of CEM connecting the main flow channel with the pre-concentration reservoirs. The reservoirs are filled with 10×TAE buffer to enhance conductance along the channel. Two platinum wires are placed in the pre-concentration reservoirs and connected to a power source (Keithley Instruments) with a constant current of 8×10⁻⁴ A. The maximum voltage applied on the pre-concentration reservoirs are 200 V to avoid possible damage to the CEM. The pre-concentration is performed for 20 min during the sample flow. Since the miRNAs are negatively charged, the positive side of the pre-concentration unit attracts the miRNAs towards the positive side, which is the opposite direction of the sample flow. When the force from the pre-concentration and the flow are balanced, the miRNAs will stay near the AEM oligoprobes. Once the pre-concentration procedure finishes, the 10×TAE buffer in the reservoirs is replaced by 0.1×PBS to avoid interference with the potentiostat measurements. Multiple high ionic washes and low ionic washes are made through the sensing channel prior to the miRNA measurements.

FIG. 3B depicts the MIX.miR board prototype (Version 1) that can detect up to three different types of miRNAs. The SAW lysis device was activated to lyse exosomes and release the enclosed miRNAs into the sample. The flow inlet allows the exosomal miRNA traveling through the pre-concentration unit and concentrating at the sensing area at the center of the flow channel. The sensing reservoir is separated into four individual compartments. Each compartment has one functionalized AEM with a specific oligoprobe for each type of miRNA. Each compartment is connected to one pair of sensing electrodes. During the experiment, the selection switch on the board is used to select the correlated miRNA, instead of unplugging the sensing electrode from the reservoir for the following reasons. First, the plugging and unplugging of the sensing electrodes may introduce bubbles in the reservoir, which will cause CVC measurement error. Second, repeated use of the sensing electrodes requires multiple calibration steps before each measurement. By using this board, only one calibration is needed in advance and the sensing unit is kept running during the CVC measurement. Finally, this also avoids possible contamination among different compartments.

The integration board was designed using commercial software PCBartist® and fabricated by the PCB manufacturer (Advanced Circuits) linked with the software. On this board, there is a green light-emitting diode (LED) for the indication of power supply, and four individual LED to indicate each miRNA target 1, 2, 3, and 4, respectively. The selection function is achieved by the encoder and decoder. The multiplexer is used to selectively connect each pair of sensing electrode to the signal interface with the potentiostat equipment. The 9 V battery and voltage regulators on the board allows for the possibility of having the board running without the presence of an external power supply. This also indicates that the board has the ability to be further miniaturized to a hand-held device for point-of-care miRNA sensing. This prototype board utilizes economic electronic parts with a total cost for one of the boards being approximately $55 ($33 for the PCB, $22 for the electronic parts, including battery). Other than the encoder, the board also contains buffers and filters to reduce electronic noise. In addition, to avoid the possible noise generated from the wiring and regulators, the layout of the board is optimized with large ground lines and non-crossing and interference signal lines. The electronic testing of the connection shows that the wiring and connection resistance between each pair of electrodes are less than 5Ω, which is significantly less than the impedance of the sensing IEM with or without miRNA.

This MIX.miR board is not a simple switch board. The binary coding shown in Table 2 provides an example of effective controlling of the multiple-target miRNA sensing. Two stages of the selection device are used to control the two parallel circuits for both the platinum electrodes (1G and 4R) and the reference electrodes (2B and 3W), as well as the LED indicators. Two bits can be used to control up to 4 different targets. If the size of the miRNA panel is to be expanded, the bit number can be increased to effectively select each target. For instance, 3 bits (a₂a₁a₀) can control 8 different miRNA targets; 4 bits (a₃a₂a₁a₀) can control 16 different miRNA targets, etc. The binary digital controlling can allow us to expand the scale of this miRNA sensing to ultra-high-throughput detection of multiple different miRNA targets simultaneously.

### Example 3

Experimental Results and Analysis of miR-1, miR-208b, and miR-499

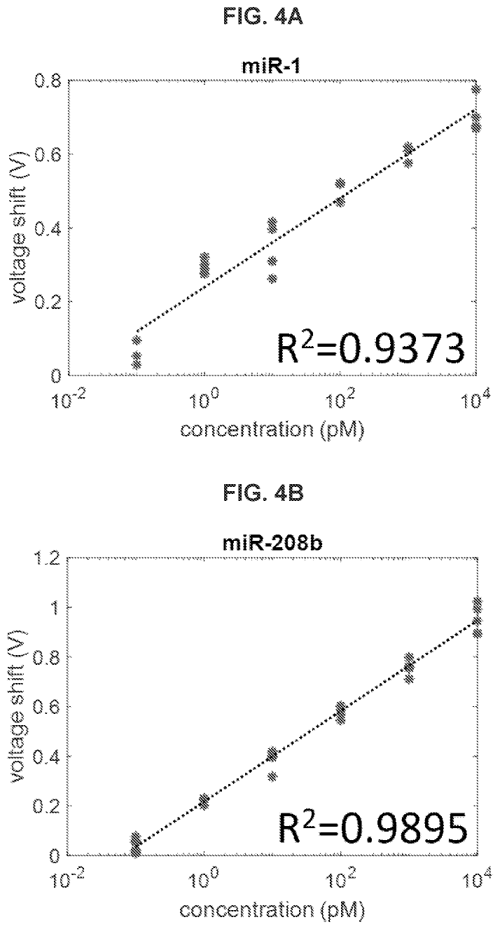

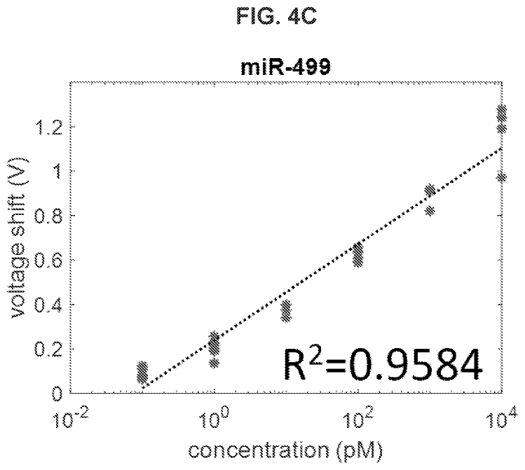

The CVC voltage shifts were correlated with the miRNA concentration through calibration curves as shown in FIG. 4A-C. The standard concentrations of 0.1 pM, 1 pM, 10 pM, 100 pM, 1 nM, and 10 nM of each miRNA were prepared (SEQ ID NO: 2; 5; 8, for miR-1, miR-208b, and miR-499 calibration oligonucleotides, respectively). The CVC voltage shifts were obtained by pre-concentration of 20 μL of each sample after a high ionic wash with 4×PBS and multiple low ionic washes with 0.1×PBS. The accuracy of MIX.miR sensors were also tested by comparing the standard concentration miRNA of 100 pM with the calibration curves.

The results of the calibration curves indicate a linear increase of the CVC voltage shifts on each decade of the concentration increments. This is consistent with earlier theories that the charged molecules change the effective surface potential that drives the electroconvective instability. The voltage shifts observed across the AEM after miRNA attachment were studied using the Langmuir adsorption model. The correlations in the linear region of the Langmuir isotherm are described by the equation:

$$\frac{V}{RT / F} = A {log}_{10} ( \frac{C}{{C}_{r}} ) ,$$

with A and reference concentration Cᵣ being (4.70, 0.0104), (7.11, 0.0651) and (8.41, 0.0786) for miR-1 (SEQ ID NO: 1), miR-208b (SEQ ID NO: 4), and miR-499 (SEQ ID NO: 7), respectively. The coefficient A is approximately the theoretical value of 2 ln₁₀(RT/F)≈0.12 V for every 10-fold decrease in bulk miRNA concentration. The constants are Faraday's constant, F=9.648×10⁴ C·mol⁻¹; the gas constant, R=8.314 J·(mol⁻¹·K⁻¹); and room temperature, T≈25° C.=298 K. These correlations are valid between the limit of detection of pM and nM with R values indicated in FIG. 4. They correspond to roughly a maximum error in the concentration estimate by a factor of 1.04, 0.438, and 0.865 for miR-1 (SEQ ID NO: 1), miR-208b (SEQ ID NO: 4), and miR-499 (SEQ ID NO: 7), respectively. The linear fitting of the calibration curve can accurately correlate the measured miRNA from clinical sample within the concentration of 0.1 pM to 10 nM. Therefore, it is preferred to work above 0.1 pM, the limit of detection (LOD).

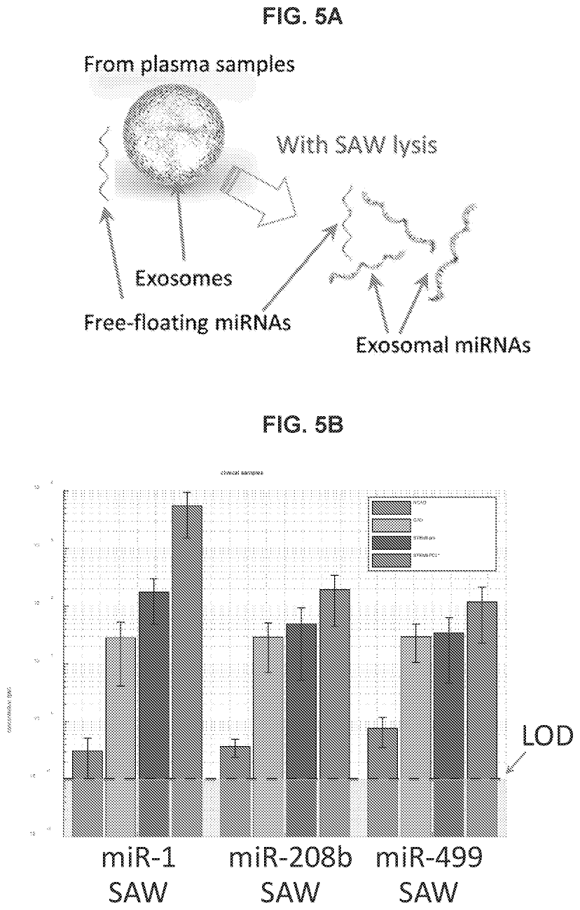

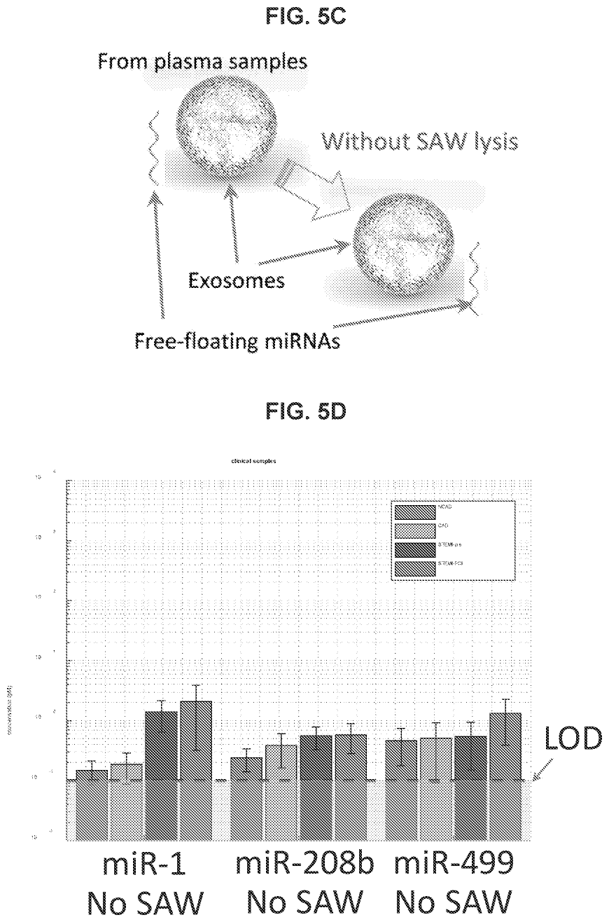

The clinical samples from subjects in the NCAD, CAD, STEMI-pre, and STEMI-PCI groups were measured individually (FIG. 5). The results from each bar graph are repeated with six different independent patient samples. Since the calibration curves of the MIX.miR sensors are testing the miRNA concentrations between 0.1 pM and 10 nM, the STEMI-post samples were diluted by 5 times in 1×PBS. The concentrations of the 3 miRNAs in STEMI-PCI samples (purple bar graph) in FIG. 5B are readings after correction for the dilution factor. The error bars represent 95% confidence levels. The uncertainty of each group is defined by:

u=t_{n-1,0.95}σ/\sqrt{n}

where n−1 is the freedom. From the measurement results in FIG. 5B with SAW lysing and those in FIG. 5D without SAW lysis, it is quite clear that the candidate miRNAs are exosomal and are only detectable after SAW lysis releases them from the nanocarrier exosomes. The MIX.miR sensor measurements in FIG. 5D indicate a low concentration around or less than 10 pM for all three different miRNAs. The miRNA concentration below 0.1 pM are below the detection limit of the MIX.miR sensor.

For the SAW lysed samples, the MIX.miR measurements revealed that the cardiac-associated miRNAs, miR-1 (SEQ ID NO: 1), miR-208b (SEQ ID NO: 4), and miR-499 (SEQ ID NO: 7) were elevated in both CAD and STEMI subjects, both pre- and post-clinical intervention. The NCAD sample provided a baseline measurement of the miRNAs. The three candidate miRNAs yield signals near the pM LOD and hence cannot be reliably detected even after SAW lysis (miR-1 (SEQ ID NO: 1): 0.31 t 0.21 pM, miR-208b (SEQ ID NO: 4): 0.37±0.12 pM, miR-499 (SEQ ID NO: 7): 0.77±0.41 pM). The CAD samples show a dramatic 10-100-fold increase in all three miRNAs (miR-1 (SEQ ID NO: 1): p=0.01, miR-208b (SEQ ID NO: 4): p=0.004, miR-499 (SEQ ID NO: 7): p=0.002). The STEMI-pre samples are the patient plasma samples before treatment and the data show a >10-fold higher expression of miR-1 (SEQ ID NO: 1) than the CAD samples, and roughly the same order of expression for miR-208 (SEQ ID NO: 4) and miR-499 (SEQ ID NO: 7) with miR-1 (SEQ ID NO: 1) (p=0.03), miR-208b (SEQ ID NO: 4) (p=0.3) and miR-499 (SEQ ID NO: 7) (p=0.7).

These results agree with current literature as the elevation of miR-499 (SEQ ID NO: 7) in AMI patient plasma is often below the detection limits of conventional miRNA quantification methods due to irregular expression. There are reports with miR-1 (SEQ ID NO: 1) and miR-208b (SEQ ID NO: 4) being overexpressed 1000 times compared to normal samples. Comparing the STEMI-pre and STEMI-PCI, the STEMI-PCI samples demonstrated an additional increase in miR-1 (SEQ ID NO: 1) and miR-208b (SEQ ID NO: 4), with miR-1 being overexpressed by another >10-fold (miR-1 (SEQ ID NO: 1): p<0.001, miR-208b (SEQ ID NO: 4): p=0.06, miR-499 (SEQ ID NO: 7): p=0.07). Consequently, miR-1 (SEQ ID NO: 1) varies significantly among CAD, STEMI-pre and STEMI-PCI samples. This finding suggests that miR-1 (SEQ ID NO: 1) is a good biomarker that can differentiate between ischemia and reperfusion injury, in addition to distinguishing STEMI patients from CAD patients. miR-208b (SEQ ID NO: 4) can differentiate STEMI and CAD patients from NCAD samples. However, the candidate miR-499 (SEQ ID NO: 7) is involved in more complicated dynamics during AMI, which regulates the mitochondrial behaviors and affects the severity of the AMI. Its relative invariance among the three groups of CAD subjects would suggest that it may be a reasonable reference control for plasma samples from patients with suspected cardiac disease.

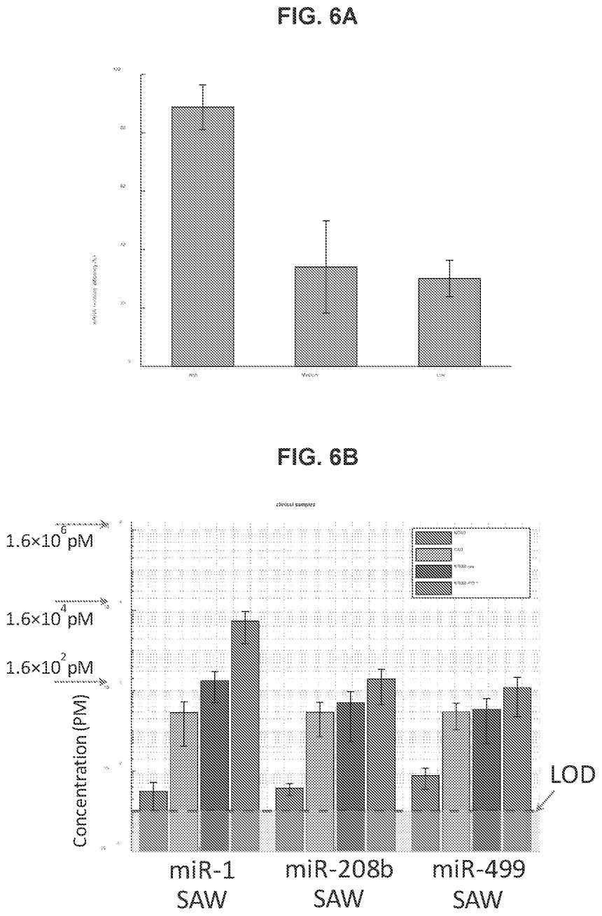

PCR quantification of three miRNAs in the clinical samples was also conducted, but only miR-1 (SEQ ID NO: 1) was detectable with the PCR tests. The other two miRNAs, miR-208b (SEQ ID NO: 7) and miR-499 (SEQ ID NO: 7), were not detectable in the same clinical samples tested with the MIX.miR after several attempts. This is likely due to loss of miRNAs during sample preparation for PCR. To evaluate the loss of miRNA during the isolation step that is necessary to perform PCR, the efficiency of miRNA isolation was measured. Efficiency was observed to be much higher (˜88%) at high concentrations of miRNA (1.6 μM) whereas at lower concentrations (16 nM and 160 pM), such as the concentrations for miR-208b and miR-499, efficiency dropped to below 50% and 40%, respectively (FIG. 6A-B). Compared with the concentrations of miR-1 (SEQ ID NO: 1), miR-208b (SEQ ID NO: 4), and miR-499 (SEQ ID NO: 7) measured by MIX.miR, the concentration of miR-1 (SEQ ID NO: 1) is much higher than miR-208b (SEQ ID NO: 4) and miR-499 (SEQ ID NO: 7). For example, the highest miR-1 (SEQ ID NO: 1) concentration in STEMI-PCI samples is within the 16 nM range, while the highest miR-208b (SEQ ID NO: 4) and miR-499 (SEQ ID NO: 7) concentrations are within the 160 pM range. During PCR, miR-208b (SEQ ID NO: 4) and miR-499 (SEQ ID NO: 7) will face a much higher loss than miR-1 (SEQ ID NO: 1). Therefore, the miR-208b (SEQ ID NO: 4) and miR-499 (SEQ ID NO: 7) are not detectable by PCR. The MIX.miR doesn't require miRNA isolation during experimentation, so there is no issue of miRNA loss.

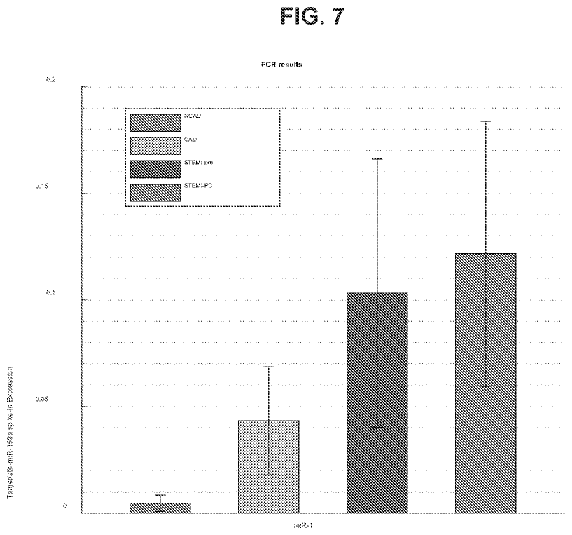

The PCR results shown in FIG. 7 indicate a significant increase in miR-1 expression in all samples when compared to NCAD. The relative overexpression level is qualitatively similar but quantitatively different from earlier miRNA RT-qPCR results in the literature, which often use a trizol or phenol/chloroform miRNA lysing/extraction technique instead of the Maxwell RSC paramagnetic bead cartridge. According to the results in miRNA isolation efficiency, the lower concentrations of miRNA have a higher loss ratio than the high concentrations of miRNA in PCR. The quantitative results of miR-1 by PCR are not fully representing the amount of miR-1 in each sample. There is also qualitative agreement with the SAW lysed MIX.miR data in FIG. 5B. However, the MIX.miR overexpression level is orders of magnitude higher, with smaller error bars. The PCR results are, however, quantitatively comparable to the MIX.miR data without SAW lysing.

The most striking of the MIX.miR results is the orders of magnitude discrepancy in overexpression of miR-1 (SEQ ID NO: 1), and the difference in the ability to detect miR-208b (SEQ ID NO: 4) and miR-499 (SEQ ID NO: 7) when comparing FIG. 5 to the RT-PCR benchmark in FIG. 7. Since the PCR quantification of miR-1 (SEQ ID NO: 1) was comparable to the non-lysed MIX⋅miR data, the majority of the miRNAs is lost due to inefficiency in miRNA extraction. This yield is expected to be even lower with chemical lysing. There are several other steps that can lead to significant RNA quantification errors. A small fraction of the extract is used in the PCR tube due to capacitance issues. The literature indicates that the extraction yield can be less than 10%, although careful benchmarking of trizol/phenol/chloroform, silica column, and paramagnetic bead extraction has not been done. From the experiments described herein, the efficiency of miRNA isolation for PCR provided an estimated loss of >60% in the samples with miRNA concentrations of 160 pM or less. Careful estimates of the miRNA reverse-transcription yield are also not available in the literature. The duplication of short length miRNA (˜22 nucleotides) may cause errors due to the heterogeneity of the exosomal miRNA mixtures and by the necessary ligation step, despite the normalization effort. Analyte loss from PCR assays due to inefficient exosome lysing, RNA extraction and reverse transcription can reduce the miRNA copy number by several orders of magnitude, severely corrupting the assay sensitivity and quantification accuracy even if PCR itself has a limit of detection of several copies. Without the need for the additional sample preparation such as miRNA isolation and reverse-transcription steps in PCR and with SAW lysing and significant reduction in sample preparation and assay time (<1-hour vs. ˜8 hours for RT-PCR), the MIX.miR sensor can hence provide a much faster assay for more biomarkers and with far higher sensitivity.

The AEM-based miRNA detection by electrical properties using fluidic devices demonstrates a potentially more efficient and accurate method for biological analysis. Extensive work has been reported using IEM in analytical chemistry. With the presence of a current across the IEM, either AEM or CEM, the trans and cis sides will become one depletion side and another enrichment side according to the concentration polarization theory. The voltage difference between the two sides will become linear with current again after reaching the over-limiting region, caused by electroconvection, water splitting, exaltation, and ion selectivity variations.

Previous work has demonstrated the ability to manipulate this phenomenon for single miRNA target detection for cancer diagnosis. Single miRNA detection of miR-146a has been reported for oral cancer, miR-21 for liver cancer, and miR-550 for pancreatic cancer with the AEM sensor. However, for many biological diagnostics, multiple kinds of miRNA targets need to be considered concurrently. In the diagnosis of AMI or CAD, a single biomarker is not necessarily sufficient as a label for differentiating specific diseases. Moreover, some evidence suggests that single miRNA biomarkers sometime fail to correlate with current clinical biomarker analysis. In addition, concurrent detection of multiple targets from the same sample can reduce the possible variations inherent in biological samples. Since the miRNAs are also sensitive to temperature once released from exosomes, detecting more miRNAs within a shorter time can also reduce the error. Furthermore, testing multiple biomarkers on a single assay would decrease the time from admission to AMI diagnosis and treatment when compared to traditional protein biomarker testing, which must be done with separate assays, increasing patient survivability. With both the multiplexing aspect of the MIX.miR and the integration board, different chambers are able to be selected for specific miRNA measurements. The digital control on the board provides us the potential to further increase the parallel quantity to enhance the multiple target sensing ability.

Recent studies have suggested miRNAs as more appropriate biomarkers for the diagnosis of AMI due to early observations of rapid changes in their expression, and have identified that miR-1 (SEQ ID NO: 1), miR-208b (SEQ ID NO: 4) and miR-499 (SEQ ID NO: 7) were among the most viable candidates for clinical use. miR-1 (SEQ ID NO: 1) is associated with the early stage of AMI that is related to the cardiac conductance; while miR-208b (SEQ ID NO: 4) and miR-499 (SEQ ID NO: 7) are associated with the late stages that regulate the expression of sarcomere contractile proteins. Studies utilizing clinical samples suggest that miR-1 (SEQ ID NO: 1) is a promising prospective biomarker for the early diagnosis of AMI to differentiate from other cardiovascular diseases. miR-208b (SEQ ID NO: 4) has also been demonstrated to show a discernible increase during AMI. However, its onset is often more delayed in AMI pathogenesis than miR-1, with detection not occurring until approximately one hour following symptom onset. Extensive clinical evidence suggests that similar to miR-1 (SEQ ID NO: 1), miR-208b (SEQ ID NO: 4) has a good correlation with the troponin assay. miR-499 (SEQ ID NO: 7) is another cardiac associated marker that is detectable in AMI, though its elevation during disease progression is not as prominent as seen for miR-1 (SEQ ID NO: 1) and miR-208b (SEQ ID NO: 4), making this increase more difficult to observe using traditional miRNA detection techniques.

Traditional methods in quantifying miRNA by RT-qPCR have trouble detecting miRNA at low concentrations due to the low efficiency of extraction during miRNA isolation and requires extensive sample preparation and processing work in miRNA isolation and amplification when compared to the MIX.miR, increasing potential error. Additionally, SAW lysis allows for the rapid (˜1 min) mechanical lysis of exosomes without additional sample preparation. SAW lysing of exosomes to release exosomal miRNA without upstream exosome isolation only makes sense if the exosomal miRNA is much more abundant than their free-floating counterparts. This is clearly evident in FIG. 5. This method is hence more efficient and simpler than chemical lysing, which involves additional chemical procedures and sample preparation steps. It also eliminates the need for a tedious exosome isolation step.

Unlike other miRNA detection methods such as RT-qPCR, the MIX.miR sensor does not require upstream sample preparation (i.e., the miRNA isolation, reverse transcription, and potential preamplification required for miRNA PCR), so that measurements may be made directly from plasma obtained within minutes of sampling. In addition, the same sample can be used to measure either free floating or free floating and exosomal miRNA through the use of surface acoustic waves (SAW), something that would require additional samples and sample preparation with other miRNA detection methods. The SAW lysis method utilizes a non-contact methodology to break the exosomes to release the exosomal miRNA with no additional chemicals. Additionally, the time from sample input to readout in the MIX.miR sensor is less than 30 minutes, significantly shorter than the thermocycling time needed for RT-qPCR. The sample volume required by the MIX.miR detection is only 20 μL; however, reliable PCR experiment requires 300-500 μL of the sample. Without the need for sample preparation and with the decrease in total assay time, the MIX.miR sensor can provide results less than an hour after collecting whole blood from a patient presenting with AMI symptoms, which is less than half the time required to obtain results from RT-qPCR. This novel approach significantly increases the viability of miRNAs as a biomarker for detection and stratification of AMI, decreasing the time from admission to date-driven clinical intervention.

The results described herein extend the current literature, with all three miRNAs showing a significant increase in concentration during STEMI prior to and following clinical intervention, as well as in patients suffering from CAD (FIG. 5). Additionally, due to its elevated sensitivity, the MIX.miR sensor was able to, for the first time, provide for the development of a method to non-invasively distinguish between the circumstance of pre-intervention coronary occlusion from that of post-intervention reperfusion through the change in circulating miR-1 (SEQ ID NO: 1) and miR-208b (SEQ ID NO: 4) expression. This distinction is currently impossible because existing biomarkers used to diagnose AMI are proteins or peptides that require longer time periods to show any discernible change in circulation and have not yet been developed as reliable markers differentiating between occlusion and reperfusion. It is anticipated that the ability of the MIX.miR sensor to detect differences in miRNA levels within minutes of reperfusion will be of great use to clinicians in guiding patient care, particularly when invasive coronary assessment is not readily available.

The multiplexing capability of MIX.miR, based on its multisensory capillary and scalable multisensory board, has allowed for the interrogation of a large panel of candidate miRNAs for AMI. In conjunction with the SAW lysing module, this multiplex platform allows for the pinpointing of miR-1 as exosomal, and its large but different overexpression for AMI and reperfusion injury suggests that this miRNA, quantified by this platform, can be used to differentiate these two conditions. The ability to quantitatively measure circulating miRNAs in concentrations as low as 1 pM in the original plasma sample with low error can potentially provide a more efficient and accurate measurement than the current biomarkers used clinically, while only using a single assay. Additionally, the relatively low cost and portability of the developed MIX.miR sensor demonstrates great potential as a rapid, specific miRNA screening tool in both clinical settings and developing countries.

### Example 4

### Additional MIX.miR Integration Board Designs

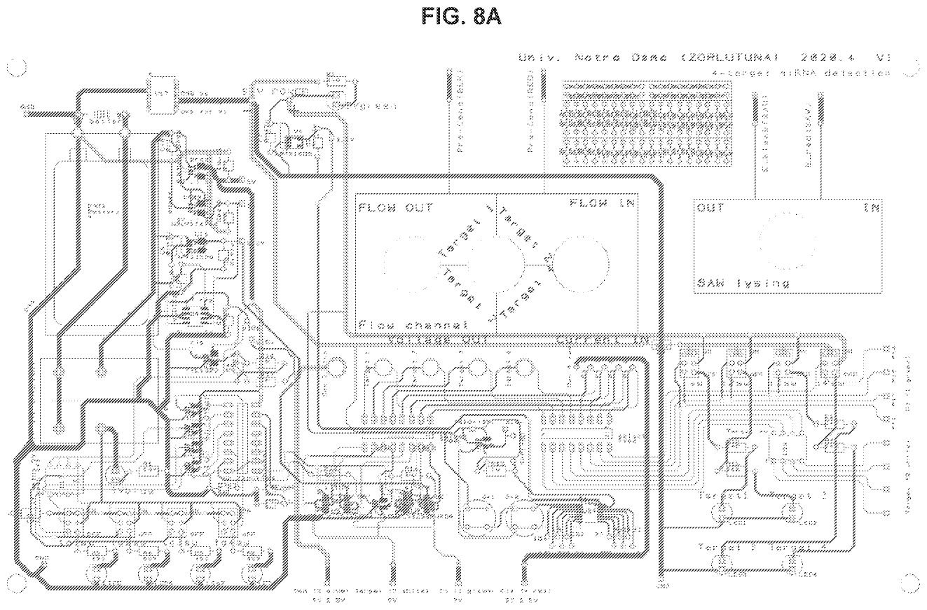

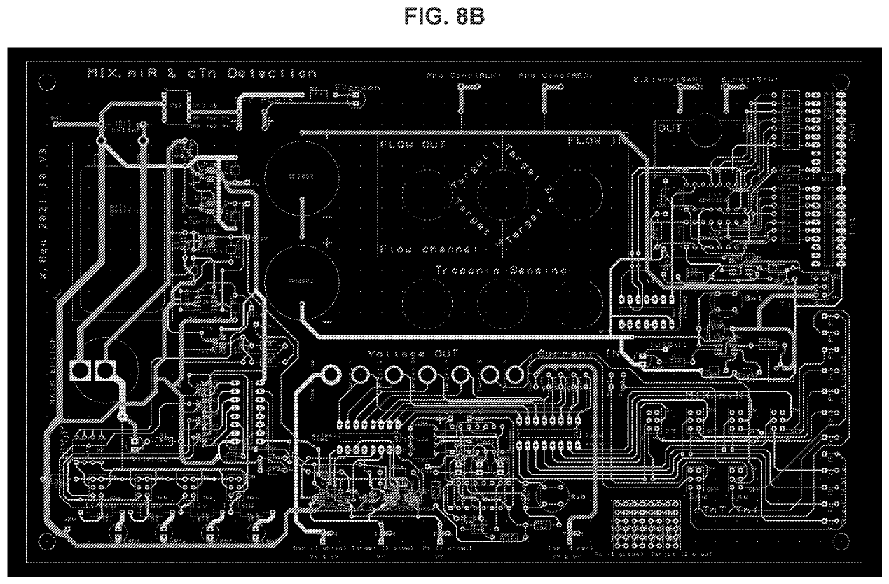

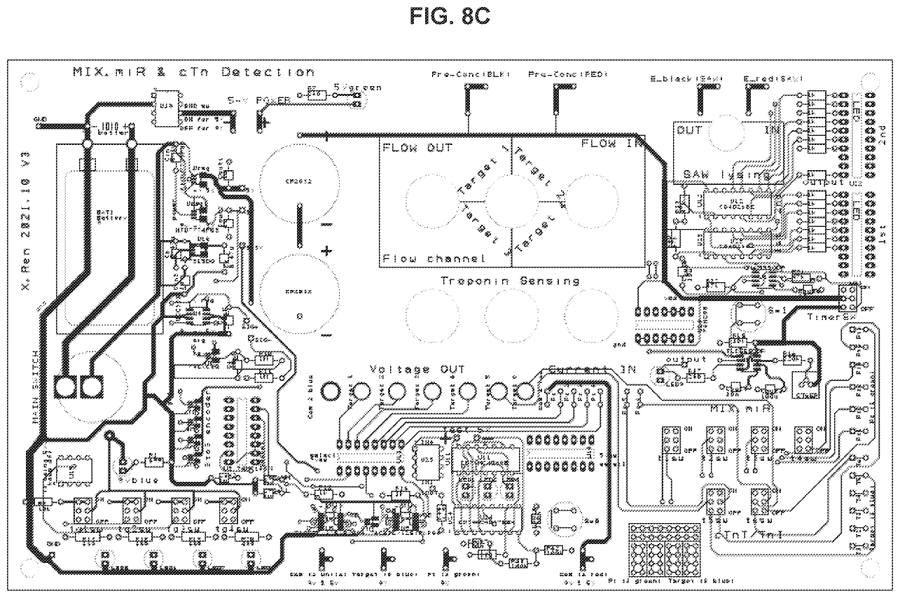

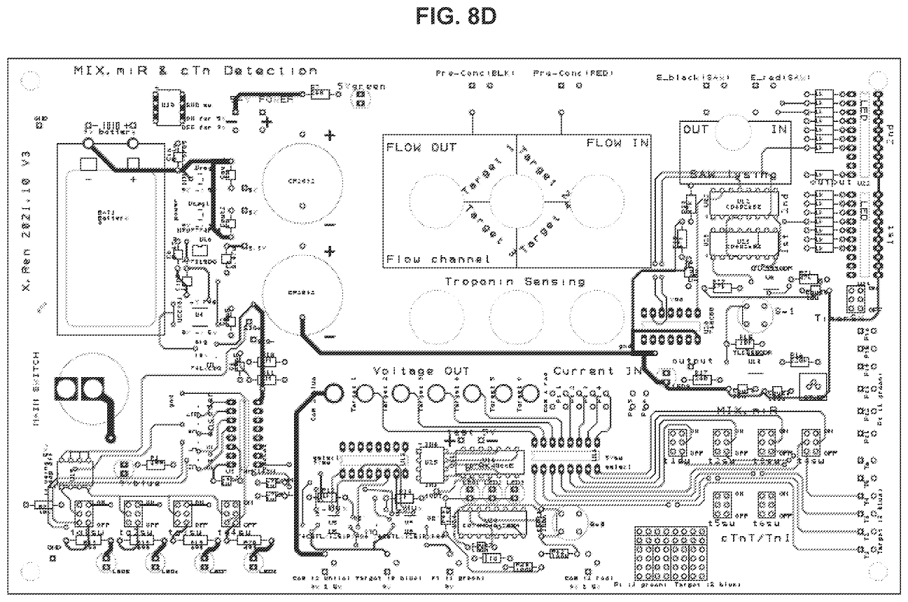

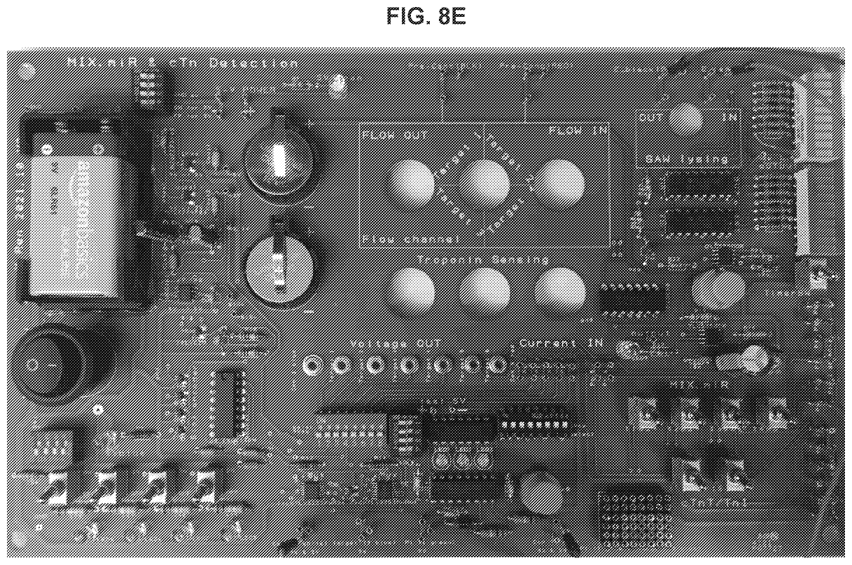

Two different versions of the MIX.miR integration board design are illustrated in FIG. 8A-E. The Version 3 MIX.miR control board (FIG. 8B-E) contains all of the same controlling modules from the Version 1 board (FIG. 8A). The Version 1 board depicted in FIG. 8A was used for Examples 1-3. The additional units of the Version 3 board include a SAW lysing timer and a simple circuit using a 555 timer. The timing is tuned by a large capacitor 100 μF and an adjustable resistor from 0-100 kΩ. The Version 3 board has the ability to hold 6 different targets at the same time, including 3-4 miRNA targets and 1-2 protein targets. For example, the board could detect 3-4 cardiac-associated miRNAs and troponin T or troponin I, which are frequently used in the diagnosis of CAD or MI.

The integration board contains a binary coding and decoding circuit to control the selection of each miRNA target. The SAW lysing device can break down exosomes in the plasma samples to release miRNAs. The SAW device was built on a piezoelectric substrate with interdigitated electrodes. The sinusoid signal for generating the SAW is 28.16 MHz with 190 mV Vpp (peak to peak voltage; Agilent 33250A, Agilent Technologies, Inc, Loveland, CO). The signal was amplified to a power of 1 W via a RF power amplifier (Electronics & Innovation 325LA RF, Rochester, NY). To maintain the integrity of the miRNAs, ice chips were placed around the SAW device and sample inlet tubing. The SAW lysing device was washed with isopropanol (IPA) and de-ionized (DI) water after each usage. The sensing electrodes are assigned with the color codes consistent with the wire color of the potentiostat (Gamry Reference 600, Gamry Instruments, PA). The electrodes 1G (green) and 4R (red) are applying current across the AEM, while 2B (blue) and 3W (white) are measuring the voltage across the AEM. The current applying on 1G and 4R is 0-8×10⁻⁵ A, with a scanning step of 1 μA/s. The three membrane chambers are sharing the common 3W and 4R. The pre-concentration unit is used to keep the negatively charged miRNAs in the sample near the AEM sensors with oligoprobes. The pre-concentration units are made by two pieces of CEM connecting the main flow channel with the pre-concentration reservoirs. The reservoirs are filled with 10×TAE buffer to enhance conductance along the channel. Two platinum wires are placed in the pre-concentration reservoirs and connected to a power source (Keithley Instruments, Cleveland, OH) with a constant current of 8×10⁻⁴ A. The maximum voltage applied on the pre-concentration reservoirs is 200 V to avoid possible damage to the CEM. The pre-concentration is performed for 20 min during the sample flow.

### Example 5

### Analysis of Subject Samples

Forty eight subjects consisting of 12 healthy subjects (normal or non-CAD, NR), 12 CAD-subject, 12 subjects diagnosed with STEMI with an observed obstruction (STEMI-pre) and 12 subjects following reperfusion (STEMI-PCI) were evaluated using the method and device described herein. The miRNA targets consisted of: miR-200b (SEQ ID NO: 10); miR-543 (SEQ ID NO: 13); miR-331 (SEQ ID NO: 16); miR-3605 (SEQ ID NO: 19); miR-301a (SEQ ID NO: 22); miR-18a (SEQ ID NO: 25); miR-423 (SEQ ID NO: 28); miR-142 (SEQ ID NO: 31); and miR-132 (SEQ ID NO: 34) as show in Table 3.

TABLE 3
miRNAs, Calibration Oligonucleotides, and Capture Oligonucleotide Sequences

| | | Calibration | Capture Oligonucleotide |
|---|---|---|---|
| miRNA | miRNA Sequence (5′→3′) | Oligonucleotide (5′→3′) | (5′→3′) |
| miR-200b | UAAUACUGCCUGGUAAUGAU | TAATACTGCCTGGTAATGAT | /5AmMC12/TCATCATTACCA |
| | GA | GA | GGCAGTATTA |
| | (SEQ ID NO: 10) | (SEQ ID NO: 11) | (SEQ ID NO: 12) |
| miR-543 | AAACAUUCGCGGUGCACUUC | AAACATTCGCGGTGCACTTC | /5AmMC12/AAGAAGTGCACC |
| | UU | TT | GCGAATGTTT |
| | (SEQ ID NO: 13) | (SEQ ID NO: 14) | (SEQ ID NO: 15) |
| miR-331 | GCCCCUGGGCCUAUCCUAGA | GCCCCTGGGCCTATCCTAGA | /5AmMC12/TTCTAGGATAGG |
| | A | A | CCCAGGGGC |
| | (SEQ ID NO: 16) | (SEQ ID NO: 17) | (SEQ ID NO: 18) |
| miR-3605 | UGAGGAUGGAUAGCAAGGAA | TGAGGATGGATAGCAAGGAA | /5AmMC12/GGCTTCCTTGCT |
| | GCC | GCC | ATCCATCCTCA |
| | (SEQ ID NO: 19) | (SEQ ID NO: 20) | (SEQ ID NO: 21) |
| miR-301a | CAGUGCAAUAGUAUUGUCAA | CAGTGCAATAGTATTGTCAA | /5AmMC12/GCTTTGACAATA |
| | AGC | AGC | CTATTGCACTG |
| | (SEQ ID NO: 22) | (SEQ ID NO: 23) | (SEQ ID NO: 24) |
| miR-18a | UAAGGUGCAUCUAGUGCAGA | TAAGGTGCATCTAGTGCAGA | /5AmMC12/CTATCTGCACTA |
| | UAG | TAG | GATGCACCTTA |
| | (SEQ ID NO: 25) | (SEQ ID NO: 26) | (SEQ ID NO: 27) |
| miR-423 | UGAGGGGCAGAGAGCGAGAC | TGAGGGGCAGAGAGCGAGAC | /5AmMC12/AAAGTCTCGCTC |
| | UUU | TTT | TCTGCCCCTCA |
| | (SEQ ID NO: 28) | (SEQ ID NO: 29) | (SEQ ID NO: 30) |
| miR-142 | CAUAAAGUAGAAAGCACUAC | CATAAAGTAGAAAGCACTAC | /5AmMC12/AGTAGTGCTTTC |
| | U | T | TACTTTATG |
| miR-132 | (SEQ ID NO: 31) | (SEQ ID NO: 32) | (SEQ ID NO: 33) |
| | UAACAGUCUACAGCCAUGGU | TAACAGTCTACAGCCATGGT | /5AmMC12/CGACCATGGCTG |
| | CG | CG | TAGACTGTTA |
| | (SEQ ID NO: 34) | (SEQ ID NO: 35) | (SEQ ID NO: 36) |

Blood was collected via standard venous puncture from healthy subjects, subjects diagnosed with CAD, and patients diagnosed with STEMI with an observed obstruction (STEMI-pre) and following reperfusion (STEMI-PCI) (12 patients for all sample groups) into tubes containing ethylenediaminetetraacetic acid (EDTA). After collection, plasma was isolated by centrifuging at 1000×g for 5 min and the plasma was transferred to RNA free tubes and stored at −80° C. and shipped to the University of Notre Dame. Once the clinical samples arrived at Notre Dame, they were thawed on ice, aliquoted and stored at −80° C. until testing with the MIX.miR sensors.

The subject demographics and results are shown in Table 4.

TABLE 4
Subject Demographics and Results

| | | | | | Hours | Troponin I |
|---|---|---|---|---|---|---|
| | Sample ID | Age | Gender | Diabetes | (pre/post) | (pg/mL) |
| NR | 007Npre/post | 47 | F | No | | |
| | 008Npre/post | 23 | F | No | | |
| | 015Npre/post | 38 | M | No | | |
| CAD | 004Cpre/post | 57 | M | | | |
| | 009Cpre/post | 73 | M | No | | |
| | 012Cpre/post | 49 | F | Yes | | |
| MIpre/ | 001Mpre/post | 55 | M | No | 2.73→3.59 | 799 |
| post | 003Mpre/post | 65 | F | No | 53.98→54.86 | NA |
| | 009Mpre/post | 80 | F | Yes | 98.15→98.7 | 3966 |
| | 010Mpre/post | 45 | M | No | 5.45→5.9 | 868 |
| | 012Mpre/post | 58 | F | No | 97.55→98.18 | <6 |
| | 014Mpre/post | 50 | M | No | 32.72→33.24 | 49 |
| NR | 012N | 63 | F | No | | |
| | 046N | 65 | F | No | | |
| | 050N | 63 | M | No | | |
| | 058N | 63 | M | No | | |
| | 066N | 67 | F | No | | |
| | 068N | 67 | M | No | | |
| CAD | 026C | 63 | M | No | | |
| | 052C | 65 | M | No | | |
| | 053C | 62 | F | No | | |
| | 054C | 65 | F | No | | |
| | 078C | 61 | F | No | | |
| | 083C | 66 | M | No | | |
| MI | 013Mpre/post | 58 | F | No | 2.57→3.29 | 8 |
| pre/ | 017Mpre/post | 50 | M | No | 1.65→2.48 | 27000 |
| post | 028Mpre/post | 67 | M | No | 5.55→6.05 | 2170 |
| | 033Mpre/post | 64 | F | No | 1.25→2.25 | 6 |
| | 034Mpre/post | 73 | M | No | 2.45→3.53 | 30 |
| | 039Mpre/post | | | | 1.27→ | |

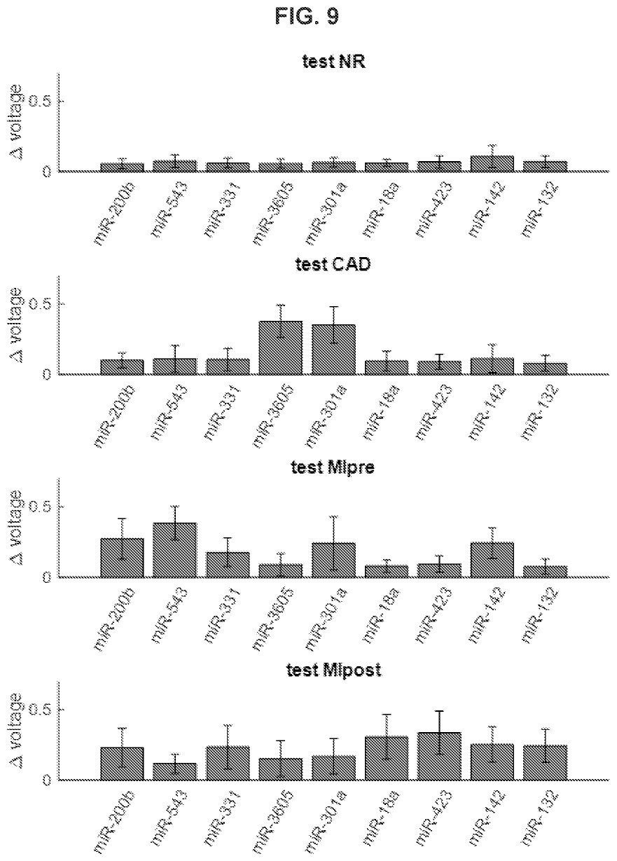

The data shown in the FIG. 9 is the direct reading of voltage shift for miR-200b (SEQ ID NO: 10); miR-543 (SEQ ID NO: 13); miR-331 (SEQ ID NO: 16); miR-3605 (SEQ ID NO: 19); miR-301a (SEQ ID NO: 22); miR-18a (SEQ ID NO: 25); miR-423 (SEQ ID NO: 28); miR-142 (SEQ ID NO: 31); and miR-132 (SEQ ID NO: 34).

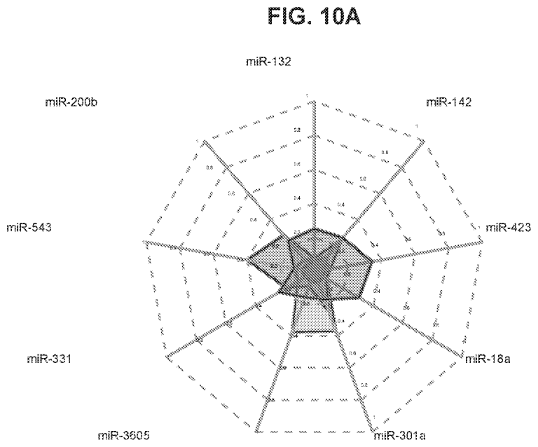

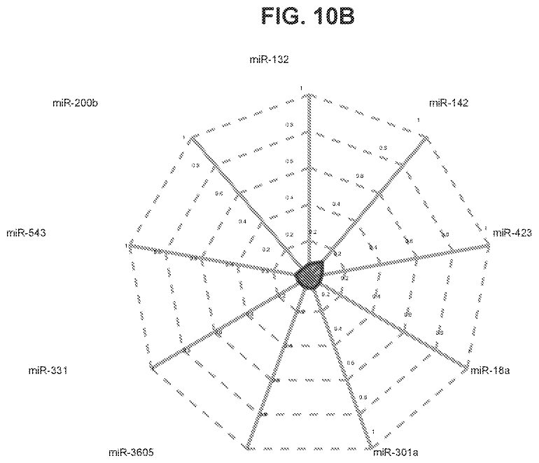

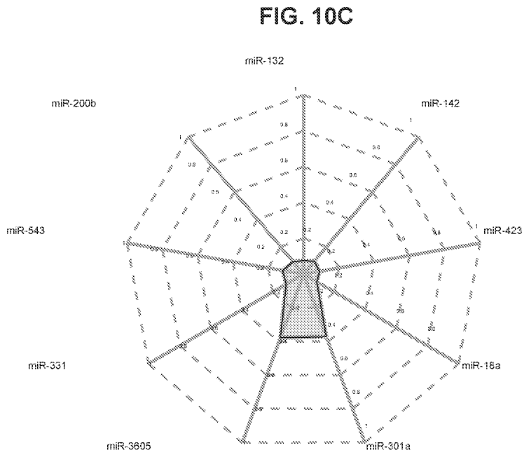

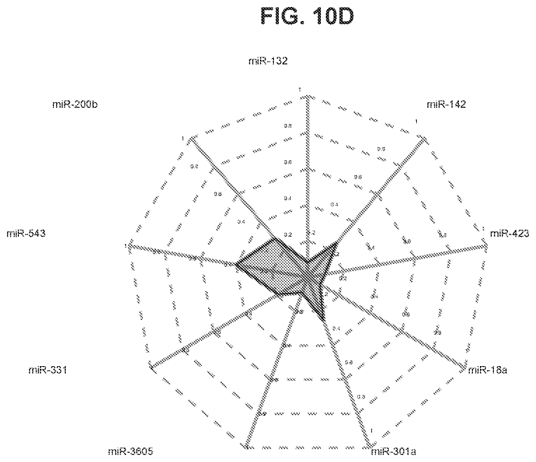

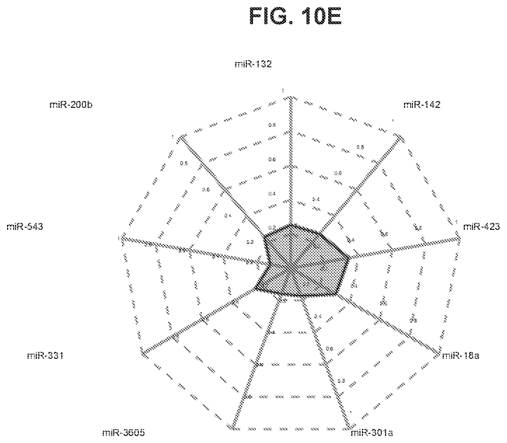

The radar maps shown in FIG. 10A-E illustrate the average voltage readings of 9 miRNAs miR-200b (SEQ ID NO: 10); miR-543 (SEQ ID NO: 13); miR-331 (SEQ ID NO: 16); miR-3605 (SEQ ID NO: 19); miR-301a (SEQ ID NO: 22); miR-18a (SEQ ID NO: 25); miR-423 (SEQ ID NO: 28); miR-142 (SEQ ID NO: 31); and miR-132 (SEQ ID NO: 34) in 4 categories (all shown in FIG. 10A): NR (blue; FIG. 10B), CAD (green; FIG. 10c), STEMI-pre (red; FIG. 10D), and STEMI-PCI (magenta; FIG. 10E).

What is claimed:

1. A method for detecting microRNAs (miRNAs) associated with acute myocardial infarction reperfusion injury, or coronary artery disease, the method comprising:
obtaining a biological sample from a subject; and
performing an assay on the biological sample to detect one or more miRNAs selected from miR-1-1 (SEQ ID NO: 1); miR-208b (SEQ ID NO: 4); miR-499 (SEQ ID NO: 7); miR-200b (SEQ ID NO: 10); miR-543 (SEQ ID NO: 13); miR-331 (SEQ ID NO: 16); miR-3605 (SEQ ID NO: 19); miR-301a (SEQ ID NO: 22); miR-18a (SEQ ID NO: 25); miR-423 (SEQ ID NO: 28); miR-142 (SEQ ID NO: 31); or miR-132 (SEQ ID NO: 34) by hybridization with oligonucleotide probes complementary to the one or more miRNAs.

2. The method of claim 1, wherein the method can distinguish between acute myocardial infarction, reperfusion injury, coronary artery disease, or normal subjects.

3. The method of claim 1, wherein the biological sample is selected from one or more of blood, serum, or plasma.

4. The method of claim 1, wherein the miRNA is free, contained in exosomes, or a combination thereof.

5. The method of claim 1, further comprising determining the concentration of the miRNAs in the biological sample and the miRNA concentrations of a control or normal subject.

6. The method of claim 5, wherein when the concentration of the miRNA in the biological sample is increased by 10-100-fold as compared to the control or normal subject, the subject is administered a treatment.

7. A method for differentiation between acute myocardial infarction reperfusion injury, or coronary artery disease, the method comprising:
obtaining a biological sample from a subject; and
performing an assay on the biological sample to detect one or more miRNAs selected from miR-1-1 (SEQ ID NO: 1); miR-208b (SEQ ID NO: 4); miR-499 (SEQ ID NO: 7); miR-200b (SEQ ID NO: 10); miR-543 (SEQ ID NO: 13); miR-331 (SEQ ID NO: 16); miR-3605 (SEQ ID NO: 19); miR-301a (SEQ ID NO: 22); miR-18a (SEQ ID NO: 25); miR-423 (SEQ ID NO: 28); miR-142 (SEQ ID NO: 31); or miR-132 (SEQ ID NO: 34) by hybridization with oligonucleotide probes complementary to the one or more miRNAs.

8. The method of claim 7, wherein the biological sample is selected from one or more of blood, serum, or plasma.

9. The method of claim 7, wherein the miRNA is free, contained in exosomes, or a combination thereof.

10. The method of claim 7, further comprising determining the concentration of the miRNAs in the biological sample and the miRNA concentrations of a control or normal subject.

11. The method of claim 10, wherein when the concentration of the miRNA in the biological sample is increased by 10-100-fold as compared to the control or normal subject, the subject is administered a treatment.

12. The method of claim 11, wherein the treatment comprises administering reperfusion therapy or a therapeutic selected from antiplatelet drugs, anticoagulants, nitrates, beta-blockers, statins, angiotensin-converting enzyme (ACE) inhibitors, angiotensin II receptor blockers (ARBs), calcium channel blockers, therapeutic hypothermia, free radical scavenger drugs, antioxidant drugs or vitamins, anesthesia, bile pigments, hydrogen (H₂), hydrogen sulfide (H₂S), nitrous oxide (NO), carbon monoxide (CO), or a combination thereof.

13. A method for detecting one or more cardiac-associated microRNAs (miRNAs), the method comprising:
obtaining a biological sample from a subject;
extracting from the biological sample a liquid component comprising exosomes and miRNA;
subjecting the liquid component to an alternating current to lyse the exosomes and release exosomal miRNAs;
concentrating the miRNAs proximate to an anion exchange membrane (AEM) functionalized with oligoprobes complementary to the miRNAs using a positively charged reservoir;
generating a current-voltage curve (CVC) of the AEM and calculating the voltage shift from the CVC of the AEM absent miRNAs;
quantifying the miRNAs bound to the oligoprobes using a calibration curve.

14. The method of claim 13, wherein the cardiac-associated miRNAs are selected from one or more of miR-1-1 (SEQ ID NO: 1); miR-208b (SEQ ID NO: 4); miR-499 (SEQ ID NO: 7); miR-200b (SEQ ID NO: 10); miR-543 (SEQ ID NO: 13); miR-331 (SEQ ID NO: 16); miR-3605 (SEQ ID NO: 19); miR-301a (SEQ ID NO: 22); miR-18a (SEQ ID NO: 25); miR-423 (SEQ ID NO: 28); miR-142 (SEQ ID NO: 31); or miR-132 (SEQ ID NO: 34).

15. The method of claim 13, wherein the miRNA is free, contained in exosomes, or a combination thereof.

16. The method of claim 13, wherein the calibration curve is generated by inputting the voltage shift (V) measured from the current-voltage curve (CVC) into the equation:
$$\frac{V}{RT / F} = A {log}_{10} ( \frac{C}{{C}_{r}} ) ,$$
wherein V is the voltage shift; R is the gas constant, 8.314 J (mol⁻¹·K⁻¹); T is the temperature, 25° C.=298 K; F is Faraday's constant, 9.648×10⁴ C mol⁻¹; A is a coefficient that is approximately the theoretical value of 2 ln₁₀(RT/F)≈0.12 V for every 10-fold decrease in bulk miRNA concentration; Cᵣ is the reference concentration; and C is the concentration of the miRNA bound to the oligoprobe.

17. The method of claim 13, further comprising determining the concentration of the miRNAs in the biological sample and the miRNA concentrations of a control or normal subject.

18. The method of claim 17, wherein when the concentration of the miRNA in the biological sample is increased by 10-100-fold as compared to the control or normal subject, the subject is administered a treatment.

19. A system for simultaneous detection of multiple distinct microRNAs (miRNAs) in a biological sample from a subject, the system comprising:
an integration board comprising:
a piezoelectric substrate comprising:
a first inlet, a first outlet, and a fluidic channel fluidly connecting the first inlet and the first outlet;
interdigitated electrodes; and
a transducer that applies alternating current to the interdigitated electrodes;
a second inlet, a second outlet, and the fluidic channel fluidly connecting the first outlet, the second inlet, and the second outlet;
one or more ion exchange arrays capable of detecting one or more distinct miRNAs, the one or more ion exchange arrays comprising:
a first cation exchange membrane (CEM) positioned across the fluidic channel proximate to the second inlet;
a second CEM positioned across the fluidic channel proximate to the second outlet;
a positively charged reservoir fluidly connected to the fluidic channel by the first CEM;
a negatively charged reservoir fluidly connected to the fluidic channel by the second CEM;
an anion exchange membrane (AEM) functionalized with a plurality of oligoprobes specific for a plurality of distinct miRNAs, fluidly connected to the fluidic channel and positioned between the first CEM and the second CEM;
at least 2 source electrodes adapted to apply current across the AEM; and
at least 2 sense electrodes adapted to measure voltage across the AEM;
a potentiostat;
a binary coding and decoding circuit to control and modulate the detection sensing for each distinct miRNA;
a plurality of probe selection switches;
a power source; and
a device for inducing fluid flow through the system.

20. The system of claim 19, wherein a calibration curve is generated by inputting the voltage shift (V) measured from a current-voltage curve (CVC) into the equation:
$$\frac{V}{RT / F} = A {log}_{10} ( \frac{C}{{C}_{r}} ) ,$$
wherein V is the voltage shift; R is the gas constant, 8.314 J (mol⁻¹·K⁻¹); T is the temperature, 25° C.=298 K; F is Faraday's constant, 9.648×10⁴ C mol⁻¹; A is a coefficient that is approximately the theoretical value of 2 ln₁₀(RT/F)≈0.12 V for every 10-fold decrease in bulk miRNA concentration; Cᵣ is the reference concentration; and C is the concentration of the miRNA bound to the oligoprobe.

21. The system of claim 19, wherein the miRNA is one or more of miR-1-1 (SEQ ID NO: 1); miR-208b (SEQ ID NO: 4); miR-499 (SEQ ID NO: 7); miR-200b (SEQ ID NO: 10); miR-543 (SEQ ID NO: 13); miR-331 (SEQ ID NO: 16); miR-3605 (SEQ ID NO: 19); miR-301a (SEQ ID NO: 22); miR-18a (SEQ ID NO: 25); miR-423 (SEQ ID NO: 28); miR-142 (SEQ ID NO: 31); or miR-132 (SEQ ID NO: 34).

22. The system of claim 19, wherein the miRNA is free, contained in exosomes, or a combination thereof.

23. The system of claim 19, wherein the system is used to determine the concentration of the miRNAs in the biological sample and the miRNA concentrations of a control or normal subject.

24. The system of claim 23, wherein when the concentration of the miRNA in the biological sample is increased by 10-100-fold as compared to the control or normal subject, the subject is administered a treatment.

25. The system of claim 19, wherein the system does not require miRNA isolation, reverse-transcription, or preamplification.

26. The system of claim 19, wherein the system detects the miRNA in from about 30 minutes to about 45 minutes.

27. The system of claim 19, wherein the system detects at least two miRNAs simultaneously.

28. The system of claim 22, wherein when the miRNA is contained in exosomes, the system lyses the exosomes in about 1 minute.

29. The system of claim 28, wherein the lysis is mechanical lysis.

30. The system of claim 19, wherein the volume of the biological sample is from about 20 μL to about 40 μL.

31. A system for simultaneous detection of multiple distinct biomolecules in a biological sample from a subject, the system comprising:
an integration board comprising:
an inlet, an outlet, and a fluidic channel fluidly connecting the inlet and the outlet;
one or more ion exchange arrays capable of detecting one or more distinct biomolecules, the one or more ion exchange arrays comprising:
a first ion exchange membrane (IEM) positioned across the fluidic channel proximate to the inlet;
a second IEM positioned across the fluidic channel proximate to the outlet;
a first charged reservoir fluidly connected to the fluidic channel by the first IEM;
a second charged reservoir fluidly connected to the fluidic channel by the second IEM;
a third IEM functionalized with a plurality of probes specific for a plurality of distinct biomolecules, fluidly connected to the fluidic channel and positioned between the first IEM and the second IEM;
a plurality of source electrodes adapted to apply current across the third IEM; and
a plurality of sense electrodes adapted to measure voltage across the third IEM;
a potentiostat;
a binary coding and decoding circuit to control and modulate the detection sensing for each of the distinct biomolecules;
a plurality of probe selection switches;
a power source; and
a device for inducing fluid flow through the system.

32. The system of claim 31, wherein the multiple distinct biomolecules are selected from nucleic acids, proteins, carbohydrates, lipids, or combinations thereof.
